(19) United States
(12) Patent Application Publication
(10) Pub. No.: US 2019/0092848 A1
(43) Pub. Date: Mar. 28, 2019

(54) Methods of treating bone diseases, disorders and/or injuries and reagents therefor

(71) Applicant: Cellmid Limited, New South Wales (AU)

(72) Inventors: Darren Jones, New South Wales (AU); Maria Halasz, New South Wales (AU); Astrid Liedert, Ulm (DE); Melanie Haffner-Luntzer, Ulm (DE); Anita Ignatius, Ulm (DE)

(21) Appl. No.: 16/081,081

(22) PCT Filed: Mar. 1, 2017
(86) PCT No.: PCT/AU2017/050179

§ 371 Date: Aug. 30, 2018

(30) Foreign Application Priority Data
Mar. 1, 2016 (AU) 2016900755

(51) Int. Cl. C07K 16/22; A61L 27/38

(52) U.S. Cl. CPC C07K 16/22; A61K 2039/545; A61L 27/3821

(57) ABSTRACT

The present disclosure relates to methods and reagents for treating bone disorders and/or increasing bone healing. In particular, present disclosure relates to the use of isolated or recombinant proteins, such as antibodies, which bind to, and inhibit or reduce the function of, midkine (hereinafter, referred to as “MK”) in the treatment of bone disorders and/or to increase bone healing.

### CROSS-REFERENCE TO RELATED APPLICATIONS

The present application claims priority from Australian Provisional Application No. 2016900755 filed on 1 Mar. 2016, the full contents of which is incorporated herein by reference.

### TECHNICAL FIELD

The present disclosure is directed to methods and reagents for treating bone disorders and/or increasing bone healing. In particular, present disclosure relates to the use of isolated or recombinant proteins, such as antibodies, which bind to, and inhibit or reduce the function of, midkine (hereinafter, referred to as “MK”) in the treatment of bone disorders and/or to increase bone healing.

### BACKGROUND

Midkine (hereinafter, referred to as “MK”) is a heparin-binding growth/differentiation factor originally found as a product of a gene transiently expressed in the process of retinoic acid-induced differentiation of embryonal carcinoma (EC) cells and is a polypeptide of 13 kDa in molecular weight rich in basic amino acids and cysteine (Kadomatsu. et al. (1988) Biochem. Biophys. Res. Commun., 1511312-1318; Tomokura et al. (1999) J. Biol. Chem, 265:10765-10770).

MK is known to have various biological activities. For example, MK expression is known to be increased in a number of different human cancer cells (Muramatsu (2002) J. Biochem. 132:359-371), and its expression has been found to promote the survival and migration of cancer cells, promote angiogenesis, and contribute to cancer progression.

MK is also known to play a central role in inflammatory processes. For example, it is known that neointimal formation after vascular injury and nephritis onset during ischemic injury in the kidney are suppressed in knockout mice deficient in MK genes. Moreover, it is also known that rheumatic injury models and post-operative adhesions are significantly suppressed in such knockout mice (WO2000/010608; WO2004/078210). Thus, MK is known to participate in inflammatory diseases such as arthritis, autoimmune disease, rheumatic arthritis (rheumatoid arthritis (RA) or osteoarthritis (OA)), multiple sclerosis, postoperative adhesion, inflammatory bowel disease, psoriasis, lupus, asthma, and neutrophil dysfunction. Furthermore, MK is known to promote the movement (migration) of inflammatory cells, such as macrophages or neutrophils. It has also been implicated in osteoclast differentiation.

The three-dimensional structure of MK has been determined by NMR and reported (Iwasaki et al. (1997) EMBO J. 16:6936-6946). MK is composed of: an N-terminal fragment (hereinafter, referred to as an “N-fragment”) consisting of amino acid residues 1 to 52; a C-terminal fragment (hereinafter, referred to as a “C-fragment”) consisting of amino acid residues 62 to 121; and a loop region (amino acid residues 53 to 61) (hereinafter, referred to as a “loop”) that links these fragments.

Each of the N- and C-fragments is mainly composed of: a portion having a three-dimensional structure consisting of three antiparallel [beta]-sheets (hereinafter, referred to as a “domain”; the domain (consisting of amino acid residues 15 to 52) in the N-fragment is referred to as an “N-domain”, and the domain (consisting of amino acid residues 62 to 104) in the C-fragment is referred to as a “C-domain”); and a terminally located portion devoid of the domain that does not assume a particular three-dimensional structure (hereinafter, referred to as a “tail”; the tail (consisting of amino acid residues 1 to 14) in the N-fragment is referred to as an “N-tail”, and the tail (consisting of amino acid residues 105 to 121) in the C-fragment is referred to as a “C-tail”).

Anti-MK antibodies against the C-domain and the N-domain are known e.g., as disclosed in WO2008/059616 and WO2012/122590 respectively. Based on previous findings that MK has a number of biological activities and is implicated in a range of diseases and conditions, these and other anti-MK antibodies may be therapeutically effective for a number of diseases/conditions.

The present disclosure relates to non-invasive treatment options for bone disorders and/or injuries. Although the treatment of long-bone fractures has improved over recent decades, the prevalence of delayed bone healing or even non-union formation remains at up to 10% (Cadet et al., (2013) J. Am. Acad. Orthop. Surg. 21:538-547; King et al., (2007) Humeral nonunion. Hand Clin. 23:449-456). The high incidence of orthopaedic complications is only partly explained by insufficient mechanical conditions for repair based on the osteosynthesis method. The majority of cases are based on other influences on fracture healing, including age or medication of the patient, pathological conditions like osteoporosis or diabetes and even genetic variations (Einhorn et al., (2015) Nature Reviews Rheumatology, 11:45-54; Hayda et al., (1998) Clin. Orthop. Relat. Res., 355(suppl):31-40). Currently, some therapeutic options are available to treat fracture-associated complications, including the application of growth factors, for example, like bone morphogenetic protein 2, bone grafts and non-invasive mechanical interventions, including low-intensity pulsed ultrasound (Einhorn T A (2003) J. Bone Joint Surg. Am. 85-A(Suppl 3):82-88; Giannoudis and Dinopoulos (2010) J. Orthop. Trauma, 24(Suppl 1):S9-16; Busse et al., (2002) CMAJ, 166:437-441). However, the success of these therapies are variable, with inconsistent results reported in the literature (Poynton et al., (2002) J. Orthop. Trauma, 24:522-525). Therefore, there remains the need for an effective, robust, well-characterized and non-invasive systemic therapy to improve fracture healing.

### SUMMARY

The present disclosure is based on the inventors' finding that treatment with an anti-MK antibody accelerated fracture healing in both young and adult mice, including mice following ovariectomy (OVX)—a recognized model for osteoporosis. In particular, the inventors have shown that administration of anti-MK antibody accelerated fracture healing through greater bone formation in the fracture callus at day 28 relative to mice not administered the anti-MK antibody. This was surprisingly evident even during the early stage of fracture healing (day 10). It was also shown that the administration of anti-MK antibody increased trabecular bone mineral density (BMD), bone volume/tissue volume (BV/TV) and thickness, and increased cortical BMD, in OVX mice. Furthermore, the inventors have shown that administration of anti-MK antibody to osteogenic cells in vitro abolished the MK-induced reduction in expression of differentiation-associated and beta-catenin-regulated genes, and decreased LRP-6 phosphorylation. Taken together, these findings provide the basis for treating bone disorders/diseases and/or injury using agents which inhibit or reduce MK activity or expression.

The present disclosure thus provides a method of promoting bone formation and/or promoting bone healing and/or increasing bone mineral density (BMD) in a subject, said method comprising administering to the subject an isolated or recombinant protein comprising an antigen binding domain of an antibody which binds specifically to MK protein.

In one example, the subject is selected from the groups consisting of:
(i) a subject with a bone fracture;
(ii) a subject who has undergone, or will undergo, a surgical procedure to create bone;
(iii) a subject who has undergone, or will undergo, a surgical procedure to promote integration of an orthopedic implant or hardware with adjacent bone; and
(iv) a subject suffering from or predisposed to osteoporosis.

In one example, administration of the isolated or recombinant protein to the subject accelerates bone healing and/or enhances osteoblast activity in the subject. For example, administration of the isolated or recombinant protein to the subject may accelerate bone healing in the subject. For example, administration of the isolated or recombinant protein to the subject may enhance osteoblast activity in the subject.

According to one example, the isolated or recombinant protein useful in the method of the disclosure binds specifically to an epitope located within the N-domain of MK and inhibits or reduces a function of MK. For example, the epitope to which the isolated or recombinant protein binds may be located within the N-domain of MK as defined by amino acid residues 1-61 of the sequence set forth in SEQ ID NO:1.

In one example, the isolated or recombinant protein useful in the method of the disclosure recognizes at least a portion of a high electrostatic potential cluster located at amino acid residues 1-61 of the sequence set forth in SEQ ID NO: 1.

In one example, the isolated or recombinant protein useful in the method of the disclosure binds specifically to a conformational epitope formed by the amino acid sequence set forth in SEQ ID NO:1, wherein the epitope includes at least two residues selected from the group consisting of 18W, 20W, 34F, 35R, 36E, 38T, 43T, 45R, 47R and 49R. In one example, the isolated or recombinant protein binds an epitope defined by residues 18W, 20W, 35R and 49R. In one example, the isolated or recombinant protein binds an epitope defined by residues 18W, 20W, 36E, 38T, 43T and 45R. In one example, the isolated or recombinant protein binds an epitope defined by residues 18W, 20W, 34F, 36E, 45R and 47R.

In one example, an isolated or recombinant protein useful in the method of the disclosure comprises:
(i) a heavy chain variable domain (V_{H}) comprising the sequence set forth in SEQ ID NO: 2 or a sequence exhibiting 95% or greater identity thereto;
(ii) a light chain variable domain (V_{L}) comprising the sequence set forth in SEQ ID NO: 3 or a sequence exhibiting 95% or greater identity thereto;
(iii) a V_{H} comprising the sequence set forth in SEQ ID NO: 2 or a sequence exhibiting 95% or greater identity thereto and a V_{L} comprising the sequence set forth in SEQ ID NO: 3 or a sequence exhibiting 95% or greater identity thereto;
(iv) a V_{H} comprising CDR1, CDR2 and CDR3 comprising the sequences set forth in SEQ ID NO: 4, 5 and 6 respectively, and a V_{L} comprising CDR1, CDR2 and CDR3 comprising the sequences set forth in SEQ ID NO: 7, 8 and 9 respectively; or
(v) three CDRs comprised within the sequence set forth in SEQ ID NO: 2 and three CDRs comprised within the sequence set forth in SEQ ID NO: 3.

In one example, an isolated or recombinant protein useful in the method of the disclosure comprises:
(i) a heavy chain variable domain (V_{H}) comprising the sequence set forth in SEQ ID NO: 10 or a sequence exhibiting 95% or greater identity thereto;
(ii) a light chain variable domain (V_{L}) comprising the sequence set forth in SEQ ID NO: 11 or a sequence exhibiting 95% or greater identity thereto;
(iii) a V_{H} comprising the sequence set forth in SEQ ID NO: 10 or a sequence exhibiting 95% or greater identity thereto and a V_{L} comprising the sequence set forth in SEQ ID NO: 11 or a sequence exhibiting 95% or greater identity thereto;
(iv) a V_{H} comprising CDR1, CDR2 and CDR3 comprising the sequences set forth in SEQ ID NO: 12, 13 and 14 respectively, and a V_{L} comprising CDR1, CDR2 and CDR3 comprising the sequences set forth in SEQ ID NO: 15, 16 and 17 respectively; or
(v) three CDRs comprised within the sequence set forth in SEQ ID NO: 10 and three CDRs comprised within the sequence set forth in SEQ ID NO: 11.

In one example, an isolated or recombinant protein useful in the method of the disclosure comprises:
(i) a heavy chain variable domain (V_{H}) comprising the sequence set forth in SEQ ID NO: 18 or a sequence exhibiting 95% or greater identity thereto;
(ii) a light chain variable domain (V_{L}) comprising the sequence set forth in SEQ ID NO: 19 or 20 or a sequence exhibiting 95% or greater identity thereto;
(iii) a V_{H} comprising the sequence set forth in SEQ ID NO: 18 or a sequence exhibiting 95% or greater identity thereto and a V_{L} comprising the sequence set forth in SEQ ID NO: 19 or 20 or a sequence exhibiting 95% or greater identity thereto;
(iv) a V_{H} comprising CDR1, CDR2 and CDR3 comprising the sequences set forth in SEQ ID NO: 21, 22 and 23 respectively, and a V_{L} comprising CDR1, CDR2 and CDR3 comprising the sequences set forth in SEQ ID NO: 24, 25 and 26 respectively; or
(v) three CDRs comprised within the sequence set forth in SEQ ID NO: 18 and three CDRs comprised within the sequence set forth in SEQ ID NO: 19 or 20.

According to another example, an isolated or recombinant protein useful in the method of the disclosure binds specifically to an epitope located within the C-domain of MK e.g., as defined by amino acid residues 62-104 of the sequence set forth in SEQ ID NO: 1, and inhibits or reduces a function of MK.

In one example, the epitope to which the isolated or recombinant protein binds is located within amino acid residues 64-73 and amino acid residues 78-101 of the sequence set forth in SEQ ID NO:1.

In another example, the epitope to which the isolated or recombinant protein binds is located within the C-domain of MK and the isolated or recombinant protein recognizes at least a portion of an epitope located at amino acid residues 64 to 66, amino acid residues 64 to 67, amino acid residues 64 to 69, amino acid residues 64 to 73, amino acid residues 84 to 96, or amino acid residues 87 to 96 of the sequence set forth in SEQ ID NO:1.

In a particular example, the isolated or recombinant protein useful in the method of the disclosure binds specifically to an epitope formed by the amino acid sequence set forth in SEQ ID NO: 1, wherein the epitope includes at least one residues selected from the group consisting of 64Y 65K, 66F, 67E, 69W, 73D, 84T, 86K, 87K 90Y and 96E.

According to any example hereof, the epitope within MK protein to which the isolated or recombinant protein of the disclosure binds may be a conformation epitope e.g., such as is an antiparallel β-sheet epitope.

In one example, isolated or recombinant protein useful in the method of the disclosure recognizes at least a portion of a high electrostatic potential cluster located at amino acid residues 62-104 of the sequence set forth in SEQ ID NO: 1. For example, the isolated or recombinant protein recognizes at least one amino acid selected from the group consisting of amino acid residues 62-64, 66, 68-70, 72, 79, 81, 85-89, 102 and 103 of the sequence set forth in SEQ ID NO:1. In one example, the isolated or recombinant protein binds specifically to an epitope formed by the amino acid sequence set forth in SEQ ID NO:1, wherein the epitope includes at least one residues selected from the group consisting of 63K, 79K, 81R 86K, 87K, 89R and 102K.

An exemplary isolated or recombinant protein useful in the method of the disclosure which binds an epitope located within the C-domain of MK comprises:
(i) a heavy chain variable domain (V_{H}) comprising CDR1, CDR2 and CDR3 comprising the sequences set forth in SEQ ID NO: 27, 28 and 29 respectively; and
(ii) a light chain variable domain (V_{L}) comprising CDR1, CDR2 and CDR3 comprising the sequences set forth in SEQ ID NO: 30, 31 and 32 respectively.

Another exemplary isolated or recombinant protein useful in the method of the disclosure which binds an epitope located within the C-domain of MK comprises:
(i) a heavy chain variable domain (V_{H}) comprising CDR1, CDR2 and CDR3 comprising the sequences set forth in SEQ ID NO: 33, 34 and 35 respectively; and
(ii) a light chain variable domain (V_{L}) comprising CDR1, CDR2 and CDR3 comprising the sequences set forth in SEQ ID NO: 36, 37 and 38 respectively.

According to any example herein, the isolated or recombinant protein comprises a heavy chain variable domain (VH) and a light chain variable domain (VL).

In one example, the VH and the VL are in a single polypeptide chain. In accordance with this example, the isolated or recombinant protein may be:

(i) a single chain Fv fragment (scFv);

(ii) a dimeric scFv (di-scFv); or

(iii) at least one of (i) and/or (ii) linked to a Fc or a heavy chain constant domain (CH) 2 and/or CH3.

In another example, the VL and VH are provided in separate polypeptide chains. In accordance with this example, the isolated or recombinant protein may be:

(i) a diabody;

(ii) a triabody;

(iii) a tetrabody;

(iv) a Fab;

(v) a F(ab′)2;

(vi) a Fv; or

(iv) one of (i) to (iii) linked to a Fc or a heavy chain constant domain (CH) 2 and/or CH3.

In one example, the isolated or recombinant protein is a chimeric, de-immunized, humanized or human antibody.

In one example, the isolated or recombinant protein may comprise a human or non-human primate heavy chain immunoglobulin constant region selected from a group consisting of IgG1, IgG2, IgG3, IgG4, IgM, IgE and IgA.

In one example, the isolated or recombinant protein may conjugated to a compound. For example, the isolated or recombinant protein may conjugated to a compound is selected from the group consisting of a radioisotope, a detectable label, a therapeutic compound, a colloid, a toxin, a nucleic acid, a peptide, a protein, a compound that increases the half-life of the protein in a subject and mixtures thereof.

The present disclosure also provides for use of an isolated or recombinant protein comprising an antigen binding domain of an antibody which binds specifically to MK protein in the preparation of a medicament for promoting bone formation and/or promoting bone healing and/or increasing bone mineral density (BMD) in a subject.

Suitable isolated or recombinant proteins are as described in any example hereof.

In one example, the medicament is for promoting bone formation and/or promoting bone healing and/or increasing BMD in a subject is selected from the groups consisting of:
(i) a subject with a bone fracture;
(ii) a subject who has undergone, or will undergo, a surgical procedure to create bone;
(iii) a subject who has undergone, or will undergo, a surgical procedure to promote integration of an orthopedic implant or hardware with adjacent bone; and
(iv) a subject suffering from or predisposed to osteoporosis.

### BRIEF DESCRIPTION OF FIGURES

FIG. 1 provides the results of biomechanical and micro-computed tomography analysis (volume of interest 1) of the fractured femurs in adult mice with and without treatment with anti-MK antibody. This figure clearly shows accelerated fracture healing in mice treated with anti-MK antibody relative to those mice administered vehicle. A) Illustrates relative flexural rigidity of the fractured femur in comparison to intact femur at day 21. B) Illustrates relative flexural rigidity of the fractured femur in comparison to intact femur at day 28. C) Illustrates bone volume to tissue volume ratio of the fracture callus at day 21. D) Illustrates bone volume to tissue volume ratio of the fracture callus at day 28. E) Illustrates tissue volume of the fracture callus at the osteotomy gap at day 21. F) Illustrates tissue volume of the fracture callus at the osteotomy gap at day 28. G) Illustrates moment of inertia of the fracture callus in bending axis x at day 21. H) Illustrates moment of inertia of the fracture callus in bending axis x at day 28. *Significantly different from vehicle (p<0.05) by Mann-Whitney-U test. (n=6-8 per group.)

FIG. 2 shows histological slices of fractured femurs analyzed for different tissue-type contents using Safranin O staining at days 10 and 21. This figures shows that administration of anti-MK antibody to mice increased bone formation in the fracture callus. A) Histomorphometric analysis of the whole callus between the two inner pin holes after 10 days of healing. B) Histomorphometric analysis of the whole callus between the two inner pin holes after 21 days of healing. C) Representative images of sections from decalcified femurs stained with Safranin O at day 10. D) Representative images of sections from decalcified femurs stained with Safranin O at day 21. Scale bar: 500 μm. *Significantly different from vehicle (p<0.05) by Mann-Whitney-U test. (n=6-8 per group.)

FIG. 3 shows sections of fractured femurs from four mice from each time point and group stained for MK and counterstained using hematoxylin. This figure shows that MK expression was decreased in areas of neovascularization and new bone formation in mice treated with anti-MK antibody. Representative images are shown; Veh=vehicle; C=cortex; V=vessels; The images on the left hand side of the upper panel show the periosteal region at day 4 after fracture with few positively stained areas. The images on the right hand side of the upper panel show the cartilaginous periosteal callus at day 10 after fracture. Mdk expression was detected in proliferating and hypertrophic chondrocytes. The images on the left hand side of the lower panel show areas of neovascularization in the bony callus at day 10 after fracture. Mdk expression was detected around the vessels in vehicle-treated, but not in Mdk-Ab treated mice. The images on the right hand side of the lower panel show areas of neovascularization in the bony callus at day 21 after fracture. Mdk expression was generally low. Scale bar: 50 μm. (n=4 per group.)

FIG. 4 illustrates that beta-catenin-positive area and osteoblast surface were increased in animals treated with anti-MK antibody relative to those administered vehicle. A) shows sections of fractured femurs from four mice of each time point and group that were stained for beta-catenin and counterstained using hematoxylin. Representative images are provided showing the periosteal callus proximal to the osteotomy gap at day 10 after fracture. Proliferating chondrocytes and osteoblasts expressed beta-catenin, whereas hypertrophic chondrocytes were negative. Beta-catenin-positive area was greater in Ab-treated mice. Scale bar: 100 μm.veh=vehicle; C=cortex; HC=hypertrophic chondrocytes; OB=osteoblasts; PC=proliferating chondrocytes. B) shows percentage beta-catenin expression in animals treated with anti-MK antibody relative to those administered vehicle (n=4 per group). C) shows the number of osteoclasts per bone perimeter in tartrate-resistant acid phosphatase stained sections of fractured femurs at day 21 for animals treated with anti-MK antibody relative to those administered vehicle. D) Toluidine-blue-stained sections were analyzed for the number of osteoblasts per bone perimeter at day 21 and E) osteoblast surface per bone surface at day 21. F) Number of osteoclasts in the periosteal callus at day 28. *Significantly different from vehicle (p>0.05) by Mann-Whitney-U test. (n=5-8 per group.)

FIG. 5 shows the results of biomechanical, μCT and histomorphometric analyses performed on fractured femurs at day 23 for OVX and sham-operated mice treated with anti-MK antibody or vehicle. This figure illustrates that treatment with anti-MK antibody accelerated osteoporotic fracture healing. Biomechanical testing: A) shows relative flexural rigidity of the fractured femur in comparison with intact femur determined by biomechanical testing. Parameters determined by μCT analysis (volume of interest 2): B) shows bone volume to tissue volume ratio, C) shows tissue volume and D) shows moment of inertia of the fracture callus in bending axis x; (n=6-7 per group). Parameters determined by histomorphometric analysis of the whole fracture callus: E) shows bone area to tissue area ratio, F) shows cartilage area to tissue area ratio and G) shows fibrous tissue area to tissue area ratio. *Significantly different from sham+vehicle or OVX+vehicle group (p<0.05). Kruskal-Wallis test. (n=5-7 per group.). H) provides representative images of sections from undecalcified femurs at day 23 stained using Giemsa; scale bar: 500 μm.

FIG. 6 illustrates that beta-catenin positive area was decreased after ovariectomy (OVX) and increased after treatment with anti-MK antibody. A) shows sections of fractured femurs from four mice of each group stained for beta-catenin and counterstained using hematoxylin. Representative images of the periosteal callus proximal to the osteotomy gap at day 10 after fracture are also shown; Veh: vehicle; C=cortex; HC=hypertrophic chondrocytes; OB=osteoblasts; PC=proliferating chondrocytes. Proliferating chondrocytes and osteoblasts expressed beta-catenin, whereas hypertrophic chondrocytes were negative for beta-catenin. Scale bar: 100 μm. B) shows percentage beta-catenin expression in animals treated with anti-MK antibody relative to those administered vehicle (n=4 per group.) *Significantly different from sham+vehicle or OVX+vehicle group (p>0.05) by Kruskal-Wallis test.

FIG. 7 illustrates that treatment with anti-MK antibody enhanced the bone content in the intact femur of ovariectomized (OVX) mice after short-time treatment. A) shows cortical bone mineral density (BMD) and B) shows cortical thickness for OVX and sham-operated mice treated with anti-MK antibody or vehicle as determined by micro-computed tomography (μCT ) analysis of the cortical bone at the midshaft of the intact femur volume of interest 3 (VOI 3). C) shows trabecular BMD, D) shows bone volume to tissue volume ratio, E) shows trabecular thickness and F) shows trabecular number for OVX and sham-operated mice treated with anti-MK antibody or vehicle as determined by μCT analysis of the distal part of the intact femur (VOI 4). *Significantly different from sham+vehicle or OVX+vehicle group (p<0.05) by Kruskal-Wallis test. (n=6-7 per group.). G) provides representative three-dimensional reconstructions of the trabecular region of the distal intact femur (VOI 4) for OVX and sham-operated mice treated with anti-MK antibody or vehicle

FIG. 8 illustrates that treatment with anti-MK antibody enhanced the bone content in vertebral bodies of ovariectomized (OVX) mice after short-time treatment. The following results are provided for OVX and sham-operated mice treated with anti-MK antibody or vehicle as determined by micro-computed tomography analysis of the vertebral bodies (volume of interest 5): A) two-dimensional images of the second caudal vertebral body, B) trabecular bone mineral density, C) trabecular bone volume to tissue volume ratio, D) trabecular thickness, and F) trabecular number. *Significantly different from sham+vehicle or OVX+vehicle group (p<0.05) by Kruskal-Wallis test. (n=6-7 per group.)

FIG. 9 illustrates that treatment of cells with anti-MK antibody diminished the negative influence of MK protein on beta-catenin signaling. A) shows percentage cell proliferation after 6 h of stimulation with MK protein and/or anti-MK antibody, normalized to the unstimulated control (dotted line) (n=6). B) shows Alpl gene expression relative to B2M expression (housekeeping gene) at day 5 of differentiation after 6 h of stimulation with MK protein and/or anti-MK antibody, normalized to the pre-differentiation values (dotted line) (n=6). C) shows cFOS and ALPL protein expression at day 5 of differentiation after 6 h of stimulation with MK protein and/or anti-MK antibody, where α-Tubulin was used as control (n=3). D) shows gene expression of cFos at day 14 of differentiation after mechanical stimulation for 30 minutes (S), incubation with MK protein for 30 minutes before mechanical stimulation (S+Mdk) and incubation with the anti-MK antibody (Mdk-Ab). Gapdh was used as the housekeeping gene and gene expression values were normalized to the unstimulated control (dotted line) (n=6-9). E) shows the results of ATDC5 and MC3T3-E1 cells incubated with or without recombinant MK protein for 1 h and immunoprecipitation performed with anti-Mdk antibody (n=6). F) shows phospho-LRP-6, LRP-6, beta-catenin and active beta-catenin protein expression in MC3T3-E1 cells after 6 h of incubation with MK protein and/or anti-MK antibody. n=3. G) shows the results of immunoprecipitation with anti-Mdk antibody using murine primary calvarian osteoblasts (PO) or primary bone marrow-derived mesenchymal stem cells (MSC). Kruskal-Wallis test. *significantly different from control values (p<0.05).

FIG. 10 illustrates the effects of recombinant MK protein and anti-MK antibody on primary osteoblasts and bone-marrow derived stem cells. A) shows cFos gene expression at day 21 of differentiation of primary osteoblasts after mechanical stimulation for 30 minutes (S), incubation with MK protein for 30 minutes before mechanical stimulation (S+Mdk) and incubation with the anti-MK antibody. Gapdh was used as the housekeeping gene and gene expression values were normalized to the unstimulated control (dotted line) (n=4-6). B) shows the level of proliferation of mMSCs (expressed as a percentage) after 6 h of stimulation with MK protein and anti-MK antibody. The expression values were normalized to the unstimulated control (dotted line) (n=6). C) shows Alpl gene expression of mMSCs at day 10 of differentiation after 6 h of stimulation with MK protein and anti-MK antibody. B2M was used as the housekeeping gene and gene expression values were normalized to the pre-differentiation values (dotted line) (n=4). Kruskal-Wallis test. *significantly different from control values (p<0.05).

FIG. 11 illustrates the influence of ovariectomy (OVX) on uterus weight, intact bone and bony callus formation. A) Uteri from sham-operated and OVX-mice 11 weeks after initial surgery. B) Weight of the uteri. C) Representative transversal μCT images of the metaphyseal region of the intact femurs. D) Representative longitudinal 3D μCT images of the fracture calli of sham-operated and OVX-mice on day 23 after fracture.

FIG. 12 shows immune-cell populations in fracture haematoma and bone marrow on day 1 after fracture as determined by flow cytometry. A-D) Percent of living B-lymphocytes (CD19⁺), inflammatory monocytes (Ly6G⁺, F4/80⁺, CD11b⁺), macrophages (Ly6G⁻, F4/80⁺, CD11b⁺), neutrophils (Ly6G⁺, F4/80⁻, CD11b⁺), T-lymphocytes (CD3⁺), cytotoxic T-lymphocytes (CD3⁺, CD8⁺) and T-helper lymphocytes (CD3⁺, CD4⁺) in the fracture haematoma and bone marrow of sham-operated (white bars) and OVX-mice (grey bars). * p<0.05, ** p<0.01, *** p<0.001 for comparison between sham and OVX-mice.

FIG. 13 shows the number of immune cells in the fracture callus on day 3 after fracture analysed by immunohistochemistry. A) Number of Ly6G-positive neutrophils per square mm of the periosteal callus. B) Number of F4/80-positive macrophages per square mm of the marrow cavity. C) Number of CD45R-positive B-lymphocytes per square mm of the periosteal callus. D) Number of CD8-positive cytotoxic T-lymphocytes per square mm of the periosteal callus. * p<0.05 for comparison between sham and OVX-mice, # p<0.05 for comparison between Mdk-Ab untreated and treated mice.

FIG. 14 provides immune-cell staining in the fracture callus on day 3 after fracture. Representative images of Ly6G-, F4/80-, CD45R- and CD6-positive cells in the fractured femurs from sham-operated and OVX-mice treated with and without Mdk-Ab. The periosteal callus proximal to the osteotomy gap is shown. N=neutrophil; B=B-lymphocyte; T=T-lymphocyte; C=cortex. Scale bar=100 μm for immunohistochemical staining and 25 μm for immunofluorescence staining.

FIG. 15 shows cytokine expression in the fracture callus on day 3 after fracture. Representative images of Mdk-, IL-6-, CCL2- and CXCL1-immunostained sections of the fractured femurs from sham-operated and OVX-mice treated with and without Mdk-Ab. The periosteal callus proximal to the osteotomy gap is shown. C=cortex. Scale bar=100 μm.

### DETAILED DESCRIPTION

Key to the Sequence Listing

SEQ ID NO: 1—Human midkine protein sequence.

SEQ ID NO:2—IP-9 variable heavy chain protein sequence.

SEQ ID NO:3—IP-9 variable light chain protein sequence.

SEQ ID NO:4—IP-9 variable heavy chain CDR1 protein sequence.

SEQ ID NO:5—IP-9 variable heavy chain CDR2 protein sequence.

SEQ ID NO:6—IP-9 variable heavy chain CDR3 protein sequence.

SEQ ID NO:7—IP-9 variable light chain CDR1 protein sequence.

SEQ ID NO:8—IP-9 variable light chain CDR2 protein sequence.

SEQ ID NO:9—IP-9 variable light chain CDR3 protein sequence.

SEQ ID NO:10—IP-10 variable heavy chain protein sequence.

SEQ ID NO:11—IP-10 variable light chain protein sequence.

SEQ ID NO:12—IP-10 variable heavy chain CDR1 protein sequence.

SEQ ID NO: 13—IP-10 variable heavy chain CDR2 protein sequence.

SEQ ID NO:14—IP-10 variable heavy chain CDR3 protein sequence.

SEQ ID NO:15—IP-10 variable light chain CDR1 protein sequence.

SEQ ID NO:16—IP-10 variable light chain CDR2 protein sequence.

SEQ ID NO: 17—IP-10 variable light chain CDR3 protein sequence.

SEQ ID NO:18—IP-13 variable heavy chain protein sequence.

SEQ ID NO:19—IP-13 variable light chain v1 protein sequence.

SEQ ID NO:20—IP-13 variable light chain v2 protein sequence.

SEQ ID NO:21—IP-13 variable heavy chain CDR1 protein sequence.

SEQ ID NO:22—IP-13 variable heavy chain CDR2 protein sequence.

SEQ ID NO:23—IP-13 variable heavy chain CDR3 protein sequence.

SEQ ID NO:24—IP-13 variable light chain CDR1 protein sequence.

SEQ ID NO:25—IP-13 variable light chain CDR2 protein sequence.

SEQ ID NO:26—IP-13 variable light chain CDR3 protein sequence.

SEQ ID NO:27—CSM-1 variable heavy chain CDR1 protein sequence.

SEQ ID NO:28—CSM-1 variable heavy chain CDR2 protein sequence.

SEQ ID NO:29—CSM-1 variable heavy chain CDR3 protein sequence.

SEQ ID NO:30—CSM-1 variable light chain CDR1 protein sequence.

SEQ ID NO:31—CSM-1 variable light chain CDR2 protein sequence.

SEQ ID NO:32—CSM-1 variable light chain CDR3 protein sequence.

SEQ ID NO:33—IP-14 variable heavy chain CDR1 protein sequence.

SEQ ID NO:34—IP-14 variable heavy chain CDR2 protein sequence.

SEQ ID NO:35—IP-14 variable heavy chain CDR3 protein sequence.

SEQ ID NO:36—IP-14 variable light chain CDR1 protein sequence.

SEQ ID NO:37—IP-14 variable light chain CDR2 protein sequence.

SEQ ID NO:38—IP-14 variable light chain CDR3 protein sequence.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., (1994) J. Mol. Biol. 242:309-320, Chothia and Lesk (1987) J. Mol. Biol. 196:901-917, Chothia et al. (1989) Nature 342:877-883, and/or Al-Lazikani et al., (1997) J. Mol. Biol. 273:927-948.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

The skilled person will be aware that an “antibody” is generally considered to be a protein that comprises a variable region made up of a plurality of immunoglobulin chains, e.g., a polypeptide comprising a V_{L} and a polypeptide comprising a V_{H}. An antibody also generally comprises constant domains, some of which can be arranged into a constant region or constant fragment or fragment crystallizable (Fc). A V_{H} and a V_{L} interact to form a Fv comprising an antigen binding region that is capable of specifically binding to one or a few closely related antigens. Generally, a light chain from mammals is either a K light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, de-immunized antibodies, non-depleting antibodies, non-activating antibodies, primatized antibodies, human antibodies and chimeric antibodies. As used herein, the term “antibody” is also intended to include formats other than full-length, intact or whole antibody molecules, such as Fab, F(ab′)2, and Fv which are capable of binding the epitopic determinant. These formats may be referred to as antibody “fragments”. These antibody formats retain some ability to selectively bind to human midkine, examples of which include, but are not limited to, the following:

(1) Fab, the fragment which contains a monovalent binding fragment of an antibody molecule and which can be produced by digestion of whole antibody with the enzyme papain to yield an intact light chain and a portion of one heavy chain;

(2) Fab′, the fragment of an antibody molecule which can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab)2 is a dimer of two Fab′ fragments held together by two disulfide bonds;

(4) Fv, defined as a genetically engineered fragment containing the variable region of the light chain and the variable region of the heavy chain expressed as two chains;

(5) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, and tetrabodies etc which may or may not be polyspecific (see, for example, WO1994/007921 and WO1998/044001); and

(6) Single domain antibody, typically a variable heavy domain devoid of a light chain.

Accordingly, an antibody in accordance with the present disclosure includes separate heavy chains, light chains, Fab, Fab′, F(ab′)2, Fc, a variable light domain devoid of any heavy chain, a variable heavy domain devoid of a light chain and Fv.

Such fragments can be produced by recombinant DNA techniques, or by enzymatic or chemical separation of intact immunoglobulins.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof. In some cases, the intact antibody may have one or more effector functions.

The antibody disclosed herein may be a humanized antibody. The term “humanized antibody”, as used herein, refers to an antibody derived from a non-human antibody, typically murine, that retains or substantially retains the antigen-binding properties of the parent antibody but which is less immunogenic in humans.

The antibody disclosed herein may be a non-depleting antibody. The term “non-depleting antibody”, as used herein, refers to an antibody that binds to its target but does not recruit the immune system's effector functions which effect target cell lysis. The immune system's effector functions are dependent on interactions of the Fc-domain with C1q, the first component of the complement cascade, and/or receptors (FcR). Complement-dependent cytotoxicity (CDC) is initiated by multiple Fc-domains interacting with C1q, which can ultimately result in lysis of target cells through the formation of the membrane attack complex (MAC). Additionally, cells of the immune system, such as granulocytes, macrophages, and NK cells, may interact via FcRs with mAbs bound to target cells. Lysis of target cells is triggered via antibody-dependent cell mediated cytotoxicity (ADCC) or phagocytosis. Non-depleting antibodies include antibody fragments without an Fc domain, including for example, monovalent (e.g., Fab, scFv, nanobodies and dAbs), bivalent (e.g., F(ab′)₂ and diabodies) and multivalent (e.g., triabodies and pentabodies) formats. In addition, non-depleting antibodies include antibodies that have been modified to remove effector functions without impacting pharmokinetics, for example, amino acid residues in the Fc-domain that play a dominant role in interaction with C1q and FcRs could be modified, or the N-linked glycosylation site in the CH2 domain could be removed. As a skilled person is aware, the chances of engineering a non-depleting antibody are linked to the constant region used to produce the antibody. An IgG3 constant region is more likely to produce a depleting antibody than an IgG1 constant region which in turn is more likely to produce a depleting antibody than an IgG2 constant region, whereas an IgG4 constant region will generally mean that the antibody is non-depleting. A skilled person would also understand that modifications to a constant region could convert a depleting antibody into a non-depleting antibody and vice versa.

The antibody disclosed herein may be a non-activating antibody. As used herein, a “non-activating antibody” refers to antibodies that bind cell surface receptors and negate or block the action of endogenous ligands.

The term “EU numbering system of Kabat” will be understood to mean the numbering of an immunoglobulin heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

As used herein, “variable region” refers to the portions of the light and/or heavy chains of an antibody as defined herein that is capable of specifically binding to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain. The amino acid positions assigned to CDRs and FRs can be defined according to Kabat (1987 and 1991, supra) or other numbering systems in the performance of methods according to the present disclosure, e.g., the hypervariable loop numbering system of Clothia and Lesk (1987 and/or 1989, supra and/or Al-Lazikani et al., 1997, supra).

As used herein, the term “complementarity determining regions” (syn. CDRs; i.e., CDR1, CDR2, and CDR3) refers to the amino acid residues of an antibody variable domain that form loops between the FRs the sequence of which vary between antibodies. Some or all of the CDRs confer the ability to bind antigen on the antibody. Each variable domain typically has three CDR regions identified as CDR1, CDR2 and CDR3. Each complementarity determining region may comprise amino acid residues from a “complementarity determining region” as defined by Kabat et al., (1991) and/or those residues from a “hypervariable loop” Chothia and Lesk (1987), or any other known numbering technique or combination thereof, including the IMGT numbering system (Lefranc et al., (2003) Dev. Comp. Immunol., 27(1)55-77).

“Framework regions” (hereinafter FR) are those variable domain residues other than the CDR residues.

The term “constant region” or “fragment crystalizable” or “Fc” or “Fc region” or “Fc portion” (which can be used interchangeably herein) as used herein, refers to a portion of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Preferably, the constant regions of the antibodies of the disclosure are derived from human immunoglobulins. Exemplary heavy chain constant regions are gamma 1 (IgG), gamma 2 (IgG2), gamma 3 (IgG3), gamma 4 (IgG4), or hybrids thereof. The light chain constant region can be of the kappa or lambda type, preferably of the kappa type.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, α and δ heavy chains comprises two constant domains.

As will be appreciated by the person skilled in the art, the term “residue” as used herein refers to an amino acid residue. Thus, the word “residue” may be used interchangeably with the term “amino acid”.

The term “recombinant” in the context of an antibody refers to the antibody when produced by a cell, or in a cell-free expression system, in an altered amount or at an altered rate compared to its native state. In one embodiment, the cell is a cell that does not naturally produce the antibody or immunoglobulin chain. However, the cell may be a cell which comprises a non-endogenous gene that causes an altered, preferably increased, amount of the polypeptide to be produced. A recombinant antibody of the disclosure includes polypeptides which have not been separated from other components of the transgenic (recombinant) cell, or cell-free expression system, in which it is produced, and an antibody produced in such cells or cell-free systems which are subsequently purified away from at least some other components.

The antibody disclosed herein may specifically bind to midkine protein (such as human midkine protein). As used herein, the term “specifically binds” shall be taken to mean a protein reacts or associates more frequently, more rapidly, with greater duration and/or with greater affinity with midkine or a specified epitope thereof than it does with alternative antigens or epitopes. As such, “specific binding” does not necessarily require exclusive binding or non-detectable binding of another antigen. The term specifically binds” is used interchangeably with “selectively binds” herein.

By “overlapping” in the context of two epitopes shall be taken to mean that two epitopes share a sufficient number of amino acid residues to permit an antibody that binds to one epitope to competitively inhibit the binding of an antibody that binds to the other epitope. For example, the two epitopes share at least 1 or 2 or 3 or 4 or 5 or 6 or more amino acids.

Reference herein to “monoclonal antibody IP-9” or to “IP-9” is a reference to the monoclonal antibody which has a variable heavy chain sequence as shown in SEQ ID NO:2 and a variable light chain sequence as shown in SEQ ID NO:3.

Reference herein to “monoclonal antibody IP-10” or to “IP-10” is a reference to the monoclonal antibody which has a variable heavy chain sequence as shown in SEQ ID NO:10 and a variable light chain sequence as shown in SEQ ID NO:11.

Reference herein to “monoclonal antibody IP-13” or to “IP-13” is a reference to the monoclonal antibody which has a variable heavy chain sequence as shown in SEQ ID NO:18 and a variable light chain sequence as shown in SEQ ID NO:19 or 20.

Reference herein to “monoclonal antibody IP-14”, “IP-14” or “murine IP-14” is a reference to the monoclonal antibody which has a variable heavy chain sequence comprising CDR1, CDR2 and CDR3 as shown in SEQ ID NOs: 33, 34 and 35, respectively, and a variable light chain sequence comprising CDR1, CDR2 and CDR3 as shown in SEQ ID NOs: 36, 37 and 38, respectively. mAb IP14 is the same antibody as designated CSM-4 in WO2008/059616.

Reference herein to “monoclonal antibody CSM-1” or “CSM-1” is a reference to the monoclonal antibody which has a variable heavy chain sequence comprising CDR1, CDR2 and CDR3 as shown in SEQ ID NOs: 27, 28 and 29, respectively, and a variable light chain sequence comprising CDR1, CDR2 and CDR3 as shown in SEQ ID NOs: 30, 31 and 32, respectively. CSM-1 is described in WO2008/059616.

As used herein, the terms “treating”, “treat” or “treatment” and variations thereof, refer to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease/condition (e.g., osteoporosis) and/or injury (e.g., bone fracture) are mitigated or eliminated or the clinical outcome or prognosis of the disease/condition or injury is improved.

As used herein, the terms “preventing”, “prevent” or “prevention” or variations thereof, refers to the provision of prophylaxis with respect to occurrence or recurrence of a disease in an individual. An individual may be predisposed to or at risk of developing the disease or disease relapse but has not yet been diagnosed with the disease or the relapse. The term prevention does not require absolute prevention but includes inhibiting the progression of the disease to some extent.

An “effective amount” refers to at least an amount effective, at dosages and for periods of time necessary, to achieve the desired therapeutic or prophylactic result. An effective amount can be provided in one or more administrations. In some examples of the present disclosure, the term “effective amount” is meant an amount necessary to effect treatment of a disease or condition as hereinbefore described. The effective amount may vary according to the disease or condition to be treated and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. The effective amount can be administered in a single dose or in a dose repeated once or several times over a treatment period.

A “therapeutically effective amount” is at least the minimum concentration required to effect a measurable improvement of a particular disease (e.g., cancer). A therapeutically effective amount herein may vary according to factors such as the disease state, age, sex, and weight of the patient, and the ability of the protein to elicit a desired response in the individual. A therapeutically effective amount is also one in which any toxic or detrimental effects of the protein are outweighed by the therapeutically beneficial effects.

A “prophylactically effective amount” refers to an amount effective, at the dosages and for periods of time necessary, to achieve the desired prophylactic result. Typically but not necessarily, since a prophylactic dose is used in mammals prior to or at an earlier stage of disease, a prophylactically effective amount may be less than a therapeutically effective amount.

The term “effective concentration 50%” (abbreviated as “EC₅₀”) represents the concentration of an antibody of the disclosure that is required for 50% of a given effect of the molecule the antibody targets (e.g. inhibiting/displacing binding of human midkine to a target thereof). It will be understood by one in the art that a lower EC₅₀ value corresponds to a more potent antibody.

The “mammal” treated according to the present disclosure may be a primate, livestock (e.g. sheep, horses, cattle, pigs, donkeys), companion animal (e.g. pets such as dogs and cats), laboratory test animal (e.g. mice, rabbits, rats, guinea pigs), performance animal (e.g. racehorses, camels, greyhounds) or captive wild animal. In one example, the mammal is a human.

Isolated or Recombinant Proteins which Bind Selectively to MK

As described herein, isolated or recombinant proteins comprising an antigen binding domain of an antibody which binds selectively to MK are contemplated for use in the method of the present disclosure. Antibodies which bind selectively to MK are known in the art, including, but not limited to, those anti-MK antibodies described in WO2008/059616, WO2012/122590 and WO2014/070642. Further anti-MK antibodies are described in Sun X. Z, et al., (1997) J. Neuropathol. Exp. Neurol. 56(12):1339-48 and Muramatsu H., et al., (2004) J. Biochem., 119:1171-77). However, other isolated or recombinant proteins suitable for use in methods of the disclosure, including anti-MK antibodies and proteins comprising antigen binding domains thereof, may be produced by methods known in the art.

Methods for producing isolated or recombinant proteins suitable for use in a method of the disclosure, including anti-MK antibodies and binding fragments thereof, are described herein. Furthermore, functional assays for determining MK binding activity of a protein and its suitability for use in a method of the disclosure are also described herein.

In one particular example, an isolated or recombinant protein contemplated for use in a method of the disclosure is protein comprising an antigen binding domain of an antibody which binds specifically to an epitope located within the N-domain of MK e.g., as defined by amino acid residues 1-61 of the sequence set forth in SEQ ID NO:1, and thereby inhibits or reduces a function of MK. For example, an isolated or recombinant protein contemplated for use in a method of the disclosure recognizes at least a portion of a high electrostatic potential cluster located at amino acid residues 1-61 of the sequence set forth in SEQ ID NO: 1.

Suitable proteins for use in a method of the disclosure may bind specifically to a conformational epitope located within the N-domain of MK formed by the amino acid sequence set forth in SEQ ID NO:1, wherein the epitope includes at least two residues selected from the group consisting of 18W, 20W, 34F, 35R, 36E, 38T, 43T, 45R, 47R and 49R. In one example, the conformational epitope is defined by residues 18W, 20W, 35R and 49R. In one example, the conformational epitope is defined by residues 18W, 20W, 36E, 38T, 43T and 45R. In one example, the conformational epitope is defined by residues 18W, 20W, 34F, 36E, 45R and 47R.

According to one particular example, an isolated or recombinant protein contemplated for use in a method of the disclosure comprises an antigen binding domain of an antibody designated IP-9 in WO2012/122590. For example, the isolated or recombinant protein may comprise:
(i) a heavy chain variable domain (V_{H}) comprising the sequence set forth in SEQ ID NO: 2 or a sequence exhibiting 95% or greater identity thereto;
(ii) a light chain variable domain (V_{L}) comprising the sequence set forth in SEQ ID NO: 3 or a sequence exhibiting 95% or greater identity thereto;
(iii) a VH comprising the sequence set forth in SEQ ID NO: 2 or a sequence exhibiting 95% or greater identity thereto and a V_{L} comprising the sequence set forth in SEQ ID NO: 3 or a sequence exhibiting 95% or greater identity thereto;
(iv) a VH comprising CDR1, CDR2 and CDR3 comprising the sequences set forth in SEQ ID NO: 4, 5 and 6 respectively, and a V_{L} comprising CDR1, CDR2 and CDR3 comprising the sequences set forth in SEQ ID NO: 7, 8 and 9 respectively; or
(v) three CDRs comprised within the sequence set forth in SEQ ID NO: 2 and three CDRs comprised within the sequence set forth in SEQ ID NO: 3.

A particularly preferred protein for use in the method of the disclosure in accordance with this example is the antibody designated IP-9 having a VH comprising the sequence set forth in SEQ ID NO: 2 and a V_{L} comprising the sequence set forth in SEQ ID NO: 3.

According to another particular example, an isolated or recombinant protein contemplated for use in a method of the disclosure comprises an antigen binding domain of an antibody designated IP-10 in WO2012/122590. For example, the isolated or recombinant protein may comprise:
(i) a heavy chain variable domain (V_{H}) comprising the sequence set forth in SEQ ID NO: 10 or a sequence exhibiting 95% or greater identity thereto;
(ii) a light chain variable domain (V_{L}) comprising the sequence set forth in SEQ ID NO: 11 or a sequence exhibiting 95% or greater identity thereto;
(iii) a V_{H} comprising the sequence set forth in SEQ ID NO: 10 or a sequence exhibiting 95% or greater identity thereto and a V_{L} comprising the sequence set forth in SEQ ID NO: 11 or a sequence exhibiting 95% or greater identity thereto;
(iv) a V_{H} comprising CDR1, CDR2 and CDR3 comprising the sequences set forth in SEQ ID NO: 12, 13 and 14 respectively, and a V_{L} comprising CDR1, CDR2 and CDR3 comprising the sequences set forth in SEQ ID NO: 15, 16 and 17 respectively; or
(v) three CDRs comprised within the sequence set forth in SEQ ID NO: 10 and three CDRs comprised within the sequence set forth in SEQ ID NO: 11.

A particularly preferred protein for use in the method of the disclosure in accordance with this example is the antibody designated IP-10 having a VH comprising the sequence set forth in SEQ ID NO: 10 and a V_{L} comprising the sequence set forth in SEQ ID NO: 11.

According to another particular example, an isolated or recombinant protein contemplated for use in a method of the disclosure comprises an antigen binding domain of an antibody designated IP-13 in WO2012/122590. For example, the isolated or recombinant protein may comprise:
(i) a heavy chain variable domain (V_{H}) comprising the sequence set forth in SEQ ID NO: 18 or a sequence exhibiting 95% or greater identity thereto;
(ii) a light chain variable domain (V_{L}) comprising the sequence set forth in SEQ ID NO: 19 or 20 or a sequence exhibiting 95% or greater identity thereto;
(iii) a V_{H} comprising the sequence set forth in SEQ ID NO: 18 or a sequence exhibiting 95% or greater identity thereto and a V_{L} comprising the sequence set forth in SEQ ID NO: 19 or 20 or a sequence exhibiting 95% or greater identity thereto;
(iv) a V_{H} comprising CDR1, CDR2 and CDR3 comprising the sequences set forth in SEQ ID NO: 21, 22 and 23 respectively, and a V_{L} comprising CDR1, CDR2 and CDR3 comprising the sequences set forth in SEQ ID NO: 24, 25 and 26 respectively; or
(v) three CDRs comprised within the sequence set forth in SEQ ID NO: 18 and three CDRs comprised within the sequence set forth in SEQ ID NO: 19 or 20.

A particularly preferred protein for use in the method of the disclosure in accordance with this example is the antibody designated IP-13 having a VH comprising the sequence set forth in SEQ ID NO: 18 and a V_{L} comprising the sequence set forth in SEQ ID NO: 19. Another particularly preferred protein for use in the method of the disclosure in accordance with this example is the antibody designated IP-13 having a VH comprising the sequence set forth in SEQ ID NO: 18 and a V_{L} comprising the sequence set forth in SEQ ID NO: 20.

Other isolated or recombinant proteins contemplated for use in a method of the disclosure comprise an antigen binding domain of an antibody which binds specifically to an epitope located within the C-domain of MK e.g., as defined by amino acid residues 62-104 of the sequence set forth in SEQ ID NO:1, and thereby inhibits or reduces a function of MK. For example, the epitope to which the isolated or recombinant protein of the disclosure binds may be located within amino acid residues 64-73 and amino acid residues 78-101 of the sequence set forth in SEQ ID NO:1. For example, the epitope to which the isolated or recombinant protein of the disclosure binds may be located at amino acid residues 64 to 66, amino acid residues 64 to 67, amino acid residues 64 to 69, amino acid residues 64 to 73, amino acid residues 84 to 96, or amino acid residues 87 to 96 of the sequence set forth in SEQ ID NO:1.

In one example, the isolated or recombinant protein may bind specifically to an epitope formed by the amino acid sequence set forth in SEQ ID NO:1, wherein the epitope includes at least one residues selected from the group consisting of 64Y 65K, 66F, 67E, 69W, 73D, 84T, 86K, 87K 90Y and 96E.

In accordance with any example described herein, the epitope within the C-domain of MK to which the isolated or recombinant protein binds may be a conformational epitope e.g., an antiparallel β-sheet epitope.

An isolated or recombinant protein contemplated for use in a method of the disclosure which comprises an antigen binding domain of an antibody which binds specifically to an epitope located within the C-domain of MK may recognize at least a portion of a high electrostatic potential cluster located at amino acid residues 62-104 of the sequence set forth in SEQ ID NO: 1. For example, the isolated or recombinant protein may recognize at least one amino acid selected from the group consisting of amino acid residues 62-64, 66, 68-70, 72, 79, 81, 85-89, 102 and 103 of the sequence set forth in SEQ ID NO: 1. In one example, the isolated or recombinant protein may bind specifically to an epitope formed by the amino acid sequence set forth in SEQ ID NO:1, wherein the epitope includes at least one residues selected from the group consisting of 63K, 79K, 81R 86K, 87K, 89R and 102K.

According to one particular example, an isolated or recombinant protein contemplated for use in a method of the disclosure comprises an antigen binding domain of an antibody designated CSM-1 in WO2008/059616. For example, the isolated or recombinant protein may comprise:
(i) a heavy chain variable domain (V_{H}) comprising CDR1, CDR2 and CDR3 comprising the sequences set forth in SEQ ID NO: 27, 28 and 29 respectively; and
(ii) a light chain variable domain (V_{L}) comprising CDR1, CDR2 and CDR3 comprising the sequences set forth in SEQ ID NO: 30, 31 and 32 respectively.

According to one particular example, an isolated or recombinant protein contemplated for use in a method of the disclosure comprises an antigen binding domain of an antibody designated CSM-1 in WO2008/059616 (herein also referred to as IP-14). For example, the isolated or recombinant protein may comprise:
(i) a heavy chain variable domain (V_{H}) comprising CDR1, CDR2 and CDR3 comprising the sequences set forth in SEQ ID NO: 33, 34 and 35 respectively; and
(ii) a light chain variable domain (V_{L}) comprising CDR1, CDR2 and CDR3 comprising the sequences set forth in SEQ ID NO: 36, 37 and 38 respectively.

The % identity of an immunoglobulin chain of an antibody disclosure herein is determined by GAP (Needleman and Wunsch, (1970) J. Mol. Biol., 48(3):443-453) analysis (GCG program) with a gap creation penalty=5, and a gap extension penalty=0.3. The query sequence is at least 50 amino acids in length, and the GAP analysis aligns the two sequences over a region of at least 50 amino acids. Even more preferably, the query sequence is at least 100 amino acids in length and the GAP analysis aligns the two sequences over a region of at least 100 amino acids. Most preferably, the two sequences are aligned over their entire length.

With regard to a defined isolated or recombinant protein of the disclosure e.g., such as an immunoglobulin chain of an antibody, it will be appreciated that % identity figures higher than those provided above will encompass preferred embodiments. Thus, where applicable, in light of the minimum % identity figures, it is preferred that the isolated or recombinant protein comprises an amino acid sequence which is at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In another embodiment, one residue is added to the nominated SEQ ID NO, one residue is deleted from the nominated SEQ ID NO, one residue is added and one residue is deleted compared to the nominated SEQ ID NO, two residues are added to the nominated SEQ ID NO, two residues are deleted from the nominated SEQ ID NO, one residue is changed from the nominated SEQ ID NO, two residues are changed from the nominated SEQ ID NO, one residue is changed and one residue is deleted from the nominated SEQ ID NO, or one residue is changed and one residue is added to the nominated SEQ ID NO, or any combination thereof.

In a preferred embodiment, there are no gaps in the alignment. More specifically, the algorithm does not need to create a gap in a contiguous stretch of amino acids to obtain an optimal (highest % identity) alignment.

Amino acid sequence mutants of the isolated or recombinant protein contemplated for use in the method of the present disclosure can be prepared by introducing appropriate nucleotide changes into a nucleic acid of the present disclosure, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide of the disclosure can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. Products derived from mutated/altered DNA can readily be screened using techniques described herein to determine if they have receptor-binding and/or -inhibitory activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the antibody and/or immunoglobulin chain molecule removed and a different residue inserted in its place. The sites of greatest interest for substitutional mutagenesis include sites identified as important for antigen binding. These sites, especially those falling within a sequence of at least three other identically conserved sites of human antibodies and/or immunoglobulin chains, are preferably substituted in a relatively conservative manner. Such conservative substitutions are shown in Table 1 under the heading of “exemplary substitutions”.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

Furthermore, if desired, unnatural amino acids or chemical amino acid analogues can be introduced as a substitution or addition into the antibody and/or immunoglobulin chain of the present disclosure. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as α-methyl amino acids, Cα-methyl amino acids, Nα-methyl amino acids, and amino acid analogues in general.

The isolated or recombinant protein contemplated for use in a method of the disclosure may comprise a heavy chain variable domain (V_{H}) and a light chain variable domain (V_{L}) of an anti-MK antibody described herein. In one example, the V_{H} and the V_{L} are provided in a single polypeptide chain. Alternatively, the V_{H} and the V_{L} may be provided in separate polypeptide chains.

According to an example in which the V_{H} and the V_{L} are provided in a single polypeptide chain, the isolated or recombinant protein of the disclosure may be provided in the form of:

(i) a single chain Fv fragment (scFv);

(ii) a dimeric scFv (di-scFv); or

(iii) at least one of (i) and/or (ii) linked to a Fc or a heavy chain constant domain (CH) 2 and/or CH3.

According to a different example in which the V_{H} and the V_{L} are provided in as separate polypeptide chains, the isolated or recombinant protein of the disclosure may be provided in the form of:

(i) a diabody;

(ii) a triabody;

(iii) a tetrabody;

(iv) a Fab;

(v) a F(ab′)2;

(vi) a Fv; or

(iv) one of (i) to (iii) linked to a Fc or a heavy chain constant domain (CH) 2 and/or CH3.

According to an example in which the isolated or recombinant protein of the disclosure is an antibody, the antibody may be a chimeric, de-immunized, humanized or human antibody.

In one example, the isolated or recombinant protein may also comprise a human or non-human primate heavy chain immunoglobulin constant region selected from a group consisting of IgG1, IgG2, IgG3, IgG4, IgM, IgE and IgA.

In a preferred embodiment, an isolated or recombinant protein described herein is an immunoglobulin light chain variable region joined directly to an immunoglobulin light chain constant region described herein. Similarly, in a further preferred embodiment an immunoglobulin heavy chain variable region described herein is joined directly to an immunoglobulin heavy chain constant region described herein.

A skilled person will understand that the variable and constant regions of an immunoglobulin heavy or light chain can be joined as described by using standard recombinant DNA technology to create a polynucleotide (encoding the joined variable and constant domains) that can be expressed in a suitable host (to produce the said immunoglobuin chain(s)) or by using peptide chemistry to synthesise the joined variable and constant domains.

In accordance with an example in which the isolated or recombinant protein is a humanised anti-MK antibody or binding fragment thereof, the humanised antibody or binding fragment will retain a significant proportion of the binding properties of the parent or precursor antibody or fragment. Suitable humanised antibodies will retain the ability to specifically bind MK protein e.g., human MK and/or mouse MK, recognized by the parent or precursor antibody used to produce such antibodies. Preferably a humanised antibody for use in a method of the disclosure exhibits substantially the same or improved binding affinity and avidity as the parent or precursor antibody. Ideally, the affinity (K_{D}) of the antibody for midkine will be greater than the parent antibody affinity for midkine.

Binding affinity can be determined by association (Ka) and dissociation (Kd) rate. Equilibrium affinity constant, K, is the ratio of Ka/Kd. Association (Ka) and dissociation (Kd) rates can be measured using surface plasmon resonance (SPR) (Rich and Myszka, (2000) Curr. Opin. Biotechnol. 11:54; Englebienne P, (1998) Analyst. 123(7):1599-1603). Instrumentation and methods for real time detection and monitoring of binding rates are known and are commercially available (BiaCore 2000, Biacore AB, Upsala, Sweden; and Malmqvist M (1999), Biochem. Soc. Trans. 27:335-340). Methods for assaying binding affinity are well known in the art and include half-maximal binding assays, competition assays, and Scatchard analysis.

As the skilled person will appreciate, “avidity” relates to the overall strength of interaction between two molecules, such as an antibody and antigen. Avidity depends on both the affinity and the valency of interactions. Furthermore, “affinity” relates to the strength of the binding between a single binding site of a molecule (e.g., an antibody) and a ligand (e.g., an antigen). The affinity of a molecule X for a ligand Y is represented by the dissociation constant (K_{d}), which is the concentration of Y that is required to occupy the combining sites of half the X molecules present in a solution. A smaller K_{d} indicates a stronger or higher affinity interaction, and a lower concentration of ligand is needed to occupy the sites.

An anti-MK antibody suitable for use in a method of the disclosure may also be a heteroconjugate antibody. Heteroconjugate antibodies are composed of two covalently joined antibodies. Such antibodies have, for example, been proposed to target immune system cells to unwanted cells (U.S. Pat. No. 4,676,980), and for treatment of HIV infection (WO1991/000360; WO1992/200373; EP 586505). It is contemplated that the antibodies may be prepared in vitro using known methods in synthetic protein chemistry, including those involving crosslinking agents.

It may be desirable to modify an antibody of the disclosure with respect to effector function, so as to enhance, e.g., the effectiveness of the antibody in treating a disorder described herein, such as osteoporosis. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC) (Caron et al., (1992) J. Exp. Med., 176(4):1191-1195; Shopes B. (1992) J. Immunol., 148(9):2918-2922). Homodimeric antibodies with enhanced activity may also be prepared using heterobifunctional cross-linkers as described in Wolff et al. (1993). Alternatively, an antibody can be engineered that has dual Fc regions and may thereby have enhanced complement lysis and ADCC capabilities (Stevenson et al., (1989) JAMA, 261:884-888).

Isolated or recombinant proteins for use in the method of the disclosure may be produced by the intervention of man e.g., as described herein. In a preferred embodiment, the isolated or recombinant protein of the disclosure is “substantially purified” or “purified”. By “substantially purified” or “purified” we mean an isolated or recombinant protein e.g., an antibody or binding fragment thereof, that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated. In another embodiment, “substantially purified” or “purified” means that the molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e. g., peptide, in the composition).

Production of Proteins which Bind MK

Isolated or recombinant proteins useful in a method of the present disclosure can be produced using methods available in the art, examples of which are described herein.

### Production of Antibodies

In one example, an isolated or recombinant protein useful in a method of the present disclosure is an antibody that binds MK. Methods for generating antibodies are known in the art and/or described in Harlow and Lane (editors) Antibodies: A Laboratory Manual, Cold Spring Harbor Laboratory, (1988). Generally, in such methods a MK protein or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

Monoclonal antibodies are one exemplary form of MK-binding protein contemplated for use in a method of the present disclosure. The term “monoclonal antibody” or “MAb” refers to a homogeneous antibody population capable of binding to the same antigen(s), for example, to the same epitope within the antigen. This term is not intended to be limited as regards to the source of the antibody or the manner in which it is made.

For the production of Mabs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human immunoglobulin proteins and, for example, do not express murine immunoglobulin proteins, can also be used to generate an antibody which is suitable for use in a method of the present disclosure (e.g., as described in WO2002/066630).

Following immunization, somatic cells with the potential for producing antibodies, specifically B lymphocytes (B cells), are selected for use in the MAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, (1996) J. Immunol. Methods. 197:85-95).

Antibodies can also be produced or isolated by screening a display library, e.g., a phage display library, e.g., as described in U.S. Pat. No. 6,300,064 and/or U.S. Pat. No. 5,885,793.

### Chimeric Antibodies

In one example, an isolated or recombinant protein useful in a method of the present disclosure is a chimeric antibody which binds MK. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Typically chimeric antibodies utilize rodent or rabbit variable regions and human constant regions, in order to produce an antibody with predominantly human domains. Methods for producing chimeric antibodies are described in, e.g., U.S. Pat. Nos. 4,816,567; and 5,807,715.

The present disclosure also contemplates the use of a chimeric immunoglobulin, e.g., in which a variable region from one species is fused to a region of a protein from another species. For example, the disclosure contemplates the use of an immunoglobulin comprising a variable region from a T cell receptor of one species fused to a T cell receptor constant domain from a separate species.

### Humanized and Human Antibodies

In one example, an isolated or recombinant protein useful in a method of the present disclosure may be humanized or human antibody or protein that binds MK.

The term “humanized antibody” shall be understood to refer to a subclass of chimeric antibodies having an antigen binding site or variable region derived from an antibody from a non-human species and the remaining antibody structure of the molecule based upon the structure and/or sequence of a human antibody. The antigen-binding site comprises the complementarity determining regions (CDRs) from the non-human antibody grafted onto appropriate FRs in the variable domains of a human antibody and the remaining regions from a human antibody. Antigen binding sites may be wild type or modified by one or more amino acid substitutions. In some instances, FR residues of the human immunoglobulin are replaced by corresponding non-human residues.

Methods for humanizing non-human antibodies or parts thereof (e.g., variable regions) are known in the art. Humanization can be performed following the method of U.S. Pat. Nos. 5,225,539, or 5,585,089. Other methods for humanizing an antibody are not excluded.

The term “human antibody” as used herein in connection with antibodies refers to antibodies having variable regions (e.g. V_{H}, V_{L}) and, optionally constant regions derived from or corresponding to sequences found in humans, e.g. in the human germline or somatic cells. The “human” antibodies can include amino acid residues not encoded by human sequences, e.g. mutations introduced by random or site directed mutations in vitro (in particular mutations which involve conservative substitutions or mutations in a small number of residues of the antibody, e.g. in 1, 2, 3, 4 or 5 of the residues of the antibody, e.g. in 1, 2, 3, 4 or 5 of the residues making up one or more of the CDRs of the antibody). These “human antibodies” do not actually need to be produced by a human, rather, they can be produced using recombinant means and/or isolated from a transgenic animal (e.g., mouse) comprising nucleic acid encoding human antibody constant and/or variable regions (e.g., as described above). Human antibodies can be produced using various techniques known in the art, including phage display libraries (e.g., as described in U.S. Pat. No. 5,885,793).

Human antibodies which recognize a selected epitope can also be generated using a technique referred to as “guided selection.” In this approach a selected non-human monoclonal antibody, e.g., a mouse antibody, is used to guide the selection of a completely human antibody recognizing the same epitope (e.g., as described in U.S. Pat. No. 5,565,332).

### De-immunized Antibodies

In another example, an isolated or recombinant protein useful in a method of the present disclosure may be a de-immunized antibody or protein which binds MK. De-immunized antibodies and proteins have one or more epitopes, e.g., B cell epitopes or T cell epitopes removed (i.e., mutated) to thereby reduce the likelihood that a mammal will raise an immune response against the antibody or protein. Methods for producing de-immunized antibodies and proteins are known in the art and described, for example, in WO2000/034317, WO2004/108158 and WO2004/064724.

Methods for introducing suitable mutations and expressing and assaying the resulting protein will be apparent to the skilled artisan based on the description herein.

### Heavy Chain Antibodies

In another example, an isolated or recombinant protein useful in a method of the present disclosure may be a heavy chain of an antibody which binds MK. Heavy chain antibodies differ structurally from many other forms of antibodies, in so far as they comprise a heavy chain, but do not comprise a light chain. Accordingly, these immunoglobulins are also referred to as “heavy chain only antibodies”. Heavy chain immunoglobulins are found in, for example, camelids and cartilaginous fish (also called IgNAR).

The variable regions present in naturally occurring heavy chain antibodies are generally referred to as “V_{HH} domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO1994/004678, and WO1997/049805.

A general description of heavy chain immunoglobulins from cartilaginous fish and the variable regions thereof and methods for their production and/or isolation and/or use is found inter alia in WO2005/118629.

### Antibody Fragments

### Single-Domain Antibodies

In some examples, an isolated or recombinant protein useful in a method of the present disclosure is or comprises a single-domain antibody (which is used interchangeably with the term “domain antibody” or “dAb”) which binds MK. A single-domain antibody is a single polypeptide chain comprising all or a portion of the heavy chain variable domain of an antibody. In certain examples, a single-domain antibody is a human single-domain antibody (Domantis, Inc., Waltham, Mass.; see, e.g., U.S. Pat. No. 6,248,516).

### Diabodies, Triabodies, Tetrabodies

In some examples, an isolated or recombinant protein which binds MK useful in a method of the present disclosure is or comprises a diabody, triabody, tetrabody or higher order protein complex such as those described in WO1998/044001 and/or WO1994/007921.

For example, a diabody is a protein comprising two associated polypeptide chains, each polypeptide chain comprising the structure V_{L}—X—V_{H} or V_{H}—X—V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

A diabody, triabody, tetrabody, etc capable of inducing effector activity can be produced using an antigen binding domain capable of binding to IL-3R and an antigen binding domain capable of binding to a cell surface molecule on an immune cell, e.g., a T cell (e.g., CD3).

Single Chain Fv (scFv) Fragments

In some examples, an isolated or recombinant protein useful in a method of the present disclosure is or comprises a scFvs fragment which binds MK. The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present disclosure also contemplates the use of a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses the use of a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

The present disclosure also contemplates the use of a dimeric scFv capable of inducing effector activity. In one example, the dimeric protein is a combination of a dAb and a scFv. Examples of bispecific antibody fragments capable of inducing effector function are described, for example, in U.S. Pat. No. 7,235,641.

### Recombinant Expression of MK-Binding Proteins

In some examples, an isolated or recombinant protein useful in a method of the present disclosure is produced by recombinant techniques.

In the case of a recombinant protein, a nucleic acid encoding same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce immunoglobulin protein. Exemplary cells used for expressing an immunoglobulin are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. Nos. 4,816,567 or 5,530,101.

Following isolation, the nucleic acid is inserted operably-linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably-linked. Exemplary promoters can contain additional copies of one or more specific regulatory elements to further enhance expression and/or alter the spatial expression and/or temporal expression of said nucleic acid.

As used herein, the term “operably-linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding an immunoglobulin (e.g., derived from the information provided herein), an enhancer element, a promoter, and a transcription termination sequence. The skilled artisan will be aware of suitable sequences for expression of an immunoglobulin. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the immunoglobulin may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Isolation/Purification of MK-Binding Proteins

MK-binding proteins used in accordance with the method of the present disclosure are preferably isolated, and, more preferably, provided in a substantially purified form. Methods for isolating and purifying antibodies and proteins are known in the art and/or described herein.

Where an antibody or antibody fragment is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibodies and antibody fragments prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO1999/057134 or Ed Harlow and David Lane (editors) Antibodies: A Laboratory Manual, Cold Spring Harbour Laboratory, (1988).

The skilled artisan will also be aware that a protein of the disclosure e.g., such as an antibody or fragment thereof, can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, an immunoglobulin comprising a hexa-his tag is purified by contacting a sample comprising the immunoglobulin with nickel-nitrilotriacetic acid (Ni-NTA) that specifically binds a hexa-his tag immobilized on a solid or semi-solid support, washing the sample to remove unbound antibodies, and subsequently eluting the bound antibodies. Alternatively, or in addition a ligand or antibody that binds to a tag may be used in an affinity purification method.

### Conjugates

Isolated or recombinant proteins used in accordance with the present disclosure may be provided as conjugates of proteins e.g., antibodies or binding fragments thereof, as described herein according to any embodiment. For example, an isolate or recombinant protein of the disclosure can be modified to contain additional non-proteinaceous moieties that are known in the art and readily available. Preferably, the moieties suitable for derivatization of the protein are physiologically acceptable polymer, preferably a water soluble polymer. Such polymers are useful for increasing stability and/or reducing clearance (e.g., by the kidney) and/or for reducing immunogenicity of a protein of the disclosure. Non-limiting examples of water soluble polymers include, but are not limited to, polyethylene glycol (PEG), polyvinyl alcohol (PVA), or propropylene glycol (PPG).

In one example, an isolated or recombinant protein as described herein according to any embodiment is conjugated or linked to another protein, including another protein of the disclosure or a protein comprising an antibody variable region, such as an antibody or a protein derived therefrom, e.g., as described herein. Other proteins are not excluded. Additional proteins will be apparent to the skilled artisan and include, for example, an immunomodulator or a half-life extending protein or a peptide or other protein that binds to serum albumin amongst others.

Exemplary serum albumin binding peptides or protein are described in US20060228364 or US20080260757.

In one example, an isolated or recombinant protein used in accordance with the present disclosure comprises one or more detectable markers to facilitate detection and/or isolation. For example, the compound comprises a fluorescent label such as, for example, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, or in addition, the isolated or recombinant protein as described herein according to any embodiment is labeled with, for example, a fluorescent semiconductor nanocrystal (as described, for example, in U.S. Pat. No. 6,306,610).

Alternatively, or in addition, the isolated or recombinant protein is labeled with, for example, a magnetic or paramagnetic compound, such as, iron, steel, nickel, cobalt, rare earth materials, neodymium-iron-boron, ferrous-chromium-cobalt, nickel-ferrous, cobalt-platinum, or strontium ferrite.

### Assaying MK-Binding Activity of a Protein

### In Vitro Functional Assays

In order to be suitable for use in a method of the disclosure, the isolated or recombinant protein must bind to MK and thereby inhibit or reduce MK activity and/or block transmission of a biological signal of MK. Accordingly, one or more functional assays may be performed to determine binding specificity and affinity of a candidate protein e.g., an antibody or fragment thereof, to human MK to thereby determine suitability of that protein for use in a method of the disclosure.

Various in vitro assays are available to assess the suitability of a recombinant or isolated protein of the disclosure, such an antibody, to bind MK protein and/or inhibit or reduce MK activity and/or treat a bone disease/disorder and/or increase bone healing.

For example, binding specificity and affinity of an antibody or binding protein to human MK may be assessed by ELISA. In this way, the dissociation constant (Kd) of a candidate antibody may be determined.

In another example, the “Kd” or “Kd value” for a candidate protein or antibody may be measured by a radiolabeled MK binding assay (RIA) to determined its suitability for use in a method of the disclosure. This assay equilibrates the test protein or antibody with a minimal concentration of radioactive MK protein in the presence of a titration series of unlabelled MK protein. Following washing to remove unbound MK protein, the amount of radioactivity is determined, which is indicative of the Kd of the test protein or antibody.

According to another example the “Kd” or “Kd value” is measured by using surface plasmon resonance assays, e.g., using BIAcore surface plasmon resonance (BIAcore, Inc., Piscataway, N.J.) with immobilized IL-3Rα.

In yet another example, a chemotaxis assay can be used to assess the ability of an isolated or recombinant protein of the disclosure to block binding of MK protein to a receptor thereof and/or inhibit function associated with binding of the MK to its receptor. These assays are based on the functional migration of cells in vitro or in vivo induced by a compound (chemoattractant). Chemotaxis can be assessed by any suitable means, for example, in an assay utilizing a 96-well chemotaxis plate, or using other art-recognized methods for assessing chemotaxis.

Generally, chemotaxis assays monitor the directional movement or migration of a suitable cell into or through a barrier (e.g., endothelium, a filter), toward increased levels of a compound, from a first surface of the barrier toward an opposite second surface. Membranes or filters provide convenient barriers, such that the directional movement or migration of a suitable cell into or through a filter, toward increased levels of a compound, from a first surface of the filter toward an opposite second surface of the filter, is monitored. In some assays, the membrane is coated with a substance to facilitate adhesion, such as ICAM-1, fibronectin or collagen. Such assays provide an in vitro approximation of cell “homing”.

For example, one can detect or measure inhibition of the migration of cells in a suitable container (a containing means), from a first chamber into or through a microporous membrane into a second chamber which contains a chemoattractant e.g., midkine protein, and an antibody to be tested, and which is divided from the first chamber by the membrane. A suitable membrane, having a suitable pore size for monitoring specific migration in response to compound, including, for example, nitrocellulose, polycarbonate, is selected. For example, pore sizes of about β-8 microns, and preferably about 5-8 microns can be used. Pore size can be uniform on a filter or within a range of suitable pore sizes.

To assess migration and inhibition of migration, the distance of migration into the filter, the number of cells crossing the filter that remain adherent to the second surface of the filter, and/or the number of cells that accumulate in the second chamber can be determined using standard techniques (e.g., microscopy and flow cytometry). In one embodiment, the cells are labeled with a detectable label (e.g., radioisotope, fluorescent label, antigen or epitope label), and migration can be assessed in the presence and absence of a candidate antibody by determining the presence of the label adherent to the membrane and/or present in the second chamber using an appropriate method (e.g., by detecting radioactivity, fluorescence, immunoassay). The extent of migration induced or inhibited can be determined relative to a suitable control (e.g., compared to background migration determined in the absence of the antibody, compared to the extent of migration induced by a second compound (i.e., a standard), compared with migration of untransfected cells induced by the antibody).

In one embodiment, a population of cells to which MK protein binds or which is capable if migrating to MK protein is placed in a chamber of a cell culture device that is in liquid communication with another chamber comprising MK protein (chemoattractant). The two chambers are separated by a suitable membrane, e.g., a membrane that mimics the extracellular matrix found in a subject. The amount of cell migration from one chamber to the other through the membrane is assessed in the presence or absence of candidate proteins or antibodies. A protein or antibody that prevents or reduces the amount of MK-mediated cell migration compared to a control sample (containing no protein or antibody) is considered to have MK inhibitory activity.

As will be apparent to the skilled artisan, methods of screening may involve detecting levels of osteoclast proliferation in vitro following incubation with a candidate protein, or the ability of a candidate protein to reduce MK-induced reduction in expression of differentiation-associated and beta-catenin-regulated genes and decrease in LRP-6 phosphorylation e.g., as described in Example 3 herein. Such methods are known in the art.

### In Vivo Functional Assays

In another example, the efficacy of an isolated or recombinant protein to inhibit or reduce MK activity or expression and its corresponding usefulness to treat a bone disorder/disease and/or injury in accordance with the method of the disclosure may be assessed using an in vivo assay.

For example, a candidate protein may be administered to a non-human mammal suffering a bone fracture e.g., such as a mouse which has undergone an osteotomy as described in Example 1. In accordance with this example, a candidate protein which binds MK and accelerates the rate of fracture healing relative to the rate of fracture healing in a control animal to which the protein has not been administered is considered suitable for treating bone injuries e.g., such as fractures, and/or increasing bone healing in a method described herein.

In another example, a candidate protein of the disclosure may be administered to a non-human mammal (e.g., murine) model of osteoporosis e.g., such as a OVX mouse model described in Example 2. In accordance with this example, a candidate protein that reduces or alleviates or improves at least one symptom associated with the osteoporosis e.g. by increasing BMD, BV/TV and/or bone thickness, in a mammalian subject as compared to the symptom prior to administration of the protein and/or in a control mammal to which the candidate protein has not been administered, is considered suitable for treating the disease or condition.

### Compositions

Suitably, in compositions or methods for administration of the isolated or recombinant proteins or conjugates of the disclosure to a mammal, the isolated or recombinant protein or conjugate is combined with a pharmaceutically acceptable carrier, diluent and/or excipient, as is understood in the art. Accordingly, one example of the present disclosure provides a pharmaceutical composition comprising the isolated or recombinant protein or conjugate thereof combined with a pharmaceutically acceptable carrier, diluent and/or excipient. Alternatively, the isolated or recombinant proteins or conjugates of this disclosure can be lyophilized for storage and reconstituted in a suitable carrier prior to use according to art-known lyophilization and reconstitution techniques.

In another example, the disclosure provides a kit comprising a pharmaceutically acceptable carrier, diluent and/or excipient suitable for combining or mixing with the isolated or recombinant protein or conjugate prior to administration to the mammal. For example, the isolated or recombinant proteins or conjugates of this disclosure can be provided in a lyophilized form for combining or mixing with a pharmaceutically acceptable carrier, diluent and/or excipient prior to administration to the mammal. In this example, the kit may further comprise instructions for use e.g., in accordance with a method of the disclosure.

In general terms, by “carrier, diluent or excipient” is meant a solid or liquid filler, binder, diluent, encapsulating substance, emulsifier, wetting agent, solvent, suspending agent, coating or lubricant that may be safely administered to any mammal, e.g., a human. Depending upon the particular route of administration, a variety of acceptable carriers, diluents or excipients, known in the art may be used, as for example described in Remington's Pharmaceutical Sciences (Mack Publishing Co. N.J. USA, 1991).

By way of example only, the carriers, diluents or excipients may be selected from a group including sugars (e.g. sucrose, maltose, trehalose, glucose), starches, cellulose and its derivatives, malt, gelatine, talc, calcium sulphate, oils inclusive of vegetable oils, synthetic oils and synthetic mono- or di-glycerides, lower alcohols, polyols, alginic acid, phosphate buffered solutions, lubricants such as sodium or magnesium stearate, isotonic saline and pyrogen-free water. For example, the carrier, diluent or excipient is compatible with, or suitable for, parenteral administration. Parenteral administration includes any route of administration that is not through the alimentary canal. Non-limiting examples of parenteral administration include injection, infusion and the like. By way of example, administration by injection includes intravenous, intra-arterial, intramuscular and subcutaneous injection. Also contemplated is delivery by a depot or slow-release formulation which may be delivered intradermally, intramuscularly and subcutaneously, for example.

### Bone-Related Diseases, Disorders and Injuries

As described herein, the present disclosure provides a method of promoting bone formation and/or promoting bone healing and/or increasing BMD in a subject, comprising administering to the subject an isolated or recombinant protein comprising an antigen binding domain of an antibody which binds specifically to MK protein, a conjugate of said isolated or recombinant protein or conjugate or a composition comprising same, as described herein. Accordingly, the method of the disclosure may be particularly suitable for treatment or prevention a bone-related disease, disorder, condition and/or injury in which it is desirous to promote bone formation and/or promote bone healing and/or increase BMD in a subject.

The subject to be treated may be selected from the following:
(i) a subject with a bone fracture;
(ii) a subject who has undergone, or will undergo, a surgical procedure to create bone;
(iii) a subject who has undergone, or will undergo, a surgical procedure to promote integration of an orthopaedic implant or hardware with adjacent bone; and
(iv) a subject suffering from or predisposed to osteoporosis.

The term “bone fracture” as used herein includes simple fractures, greenstick fractures, compound fractures, comminuted (multifragmentary) fractures, impacted fractures, complicated fractures, hairline fractures, compression fractures, fatigue fractures and/or pathological fractures. Examples of bone fractures that may be advantageously treated by the method of the disclosure include, but are not limited to, fractures of the spine, leg and arm. A further example of a fracture that is advantageously treated in accordance with the present disclosure is a vertebral compression fracture. Such fracture occurs when one or more of the bones of the vertebral column fractures or collapses, typically when the vertebrae are already weakened for instance as a result of ageing or a disease that weakens bone, such as osteoporosis, Paget's disease or bone cancer.

As used herein, a “surgical procedure to create bone” includes a surgical procedure to repair a fracture, a surgical procedure used to fuse vertebral bones (e.g. spine fusion), or a surgical procedure that includes, for example, integration of an implant during total joint arthroplasty, bone screws used during fracture repair, bone screws used to anchor tendons or ligaments, or any orthopedic hardware designed to mechanically stabilize the orthopedic surgical site. Similarly, a “surgical procedure to promote integration of an orthopedic implant or hardware with adjacent bone” includes a surgical procedure that includes integration of an implant during total joint arthroplasty, bone screws used during fracture repair, bone screws used to anchor tendons or ligaments, or any orthopedic hardware designed to mechanically stabilize the orthopedic surgical site.

As described herein, administration of the isolated or recombinant protein of the disclosure has been shown to increase BMD, BV/TV and thickness in mice, including OVX mice. Accordingly, the method of the disclosure may be particularly useful for treating a subject suffering from, or who is predisposed to, osteoporosis. The osteoporosis may be primary osteoporosis, secondary osteoporosis, osteogenesis imperfect or idiopathic juvenile osteoporosis.

In the case of secondary osteoporosis, the osteoporotic symptoms may be the result of another condition e.g., hyperparathyroidism, hyperthyroidism, leukemia or advanced cancer, or the result of treatment for another condition which causes bone loss or breakdown e.g., corticosteroids, thyroid replacement therapy or aromatase inhibitors (as used in treatment of breast cancer). Accordingly, the method of the disclosure may be used as a combination therapy to prevent bone loss or breakdown and/or prevent bone breakage in subjects that may become susceptible thereto.

### Dosages and Regimens

For the prevention or treatment of a bone-related disease, disorder and/or injury as described herein, the appropriate dosage of an active agent (e.g., a protein or conjugate of the disclosure), will depend on the specific disease, disorder and/or injury to be treated, the severity and course of the disease, disorder and/or injury, whether the active agent is administered for preventive or therapeutic purposes, previous therapy received by the patient, the patient's clinical history and response to the active agent, and the discretion of the attending physician. Typically, a therapeutically effective amount of the MK-binding protein or conjugate will be administered. The phrase “a therapeutically effective amount” refers to an amount sufficient to promote, induce, and/or enhance treatment or other therapeutic effect in the subject being treated. The therapeutically effective amount should be large enough to produce the desired effect but should not be so large as to cause adverse side effects. The particular dosage regimen, i.e., dose, timing, and repetition, will depend on the particular individual and that individual's medical history as assessed by a physician. Typically, a clinician will administer an active agent (e.g., MK-binding protein or conjugate comprising same) until a dosage is reached that achieves the desired result.

Generally, the dosage will vary with the age, condition, sex and extent of the disease, disorder and/or injury in the patient and can be determined by one of skill in the art. The dosage can be adjusted by the individual physician in the event of any complication. For in vivo administration of the MK-binding proteins or conjugates described herein, normal dosage amounts may vary from about 10 ng/kg up to about 100 mg/kg of an individual's body weight or more per day. Exemplary dosages and ranges thereof are described herein. For repeated administrations over several days or longer, depending on the severity of the disease, disorder and/or injury to be treated, the treatment can be sustained until a desired suppression of symptoms or treatment is achieved.

In some examples, a MK-binding protein or conjugate as described herein is administered at an initial (or loading) dose of between about 1 mg/kg to about 30 mg/kg, such as from about 1 mg/kg to about 10 mg/kg, or about 2 mg/kg or about 3 mg/kg or 4 mg/kg or 5 mg/kg. The MK-binding protein or conjugate can then be administered at a maintenance dose of between about 0.0001 mg/kg to about 1 mg/kg, such as from about 0.0005 mg/kg to about 1 mg/kg, for example, from about 0.001 mg/kg to about 1 mg/kg, such as about 0.005 mg/kg to about 1 mg/kg, for example from about 0.1 mg/kg to about 1 mg/kg, such as about 0.2 mg/kg or 0.3 mg/kg or 0.4 mg/kg or 0.5 mg/kg. The maintenance doses may be administered every 7-30 days, such as, every 10-15 days, for example, every 10 or 11 or 12 or 13 or 14 or 15 days.

Dosages for a particular MK-binding protein or conjugate may be determined empirically in mammals who have been given one or more administrations of the respective MK-binding protein or conjugate. To assess efficacy of a dosage of MK-binding protein or conjugate of the disclosure, a clinical symptom of a disease, condition or injury being treated e.g., osteoporosis and/or bone fracture, can be monitored following administration. For example, efficacy of a dosage of MK-binding protein or conjugate of the disclosure in treatment of osteoporosis may be assessed based on BMD of a patient following treatment using a test known in the art e.g., μCT (Micro-computed tomography) or Dexa-Scan (Dual-energy X-ray absorptiometry or DEXA). In another example, efficacy of a dosage of MK-binding protein or conjugate of the disclosure in treatment of a fracture i.e., by assessing stage of fracture healing and/or facture union, may be assessed by radiography or other method known in the art.

Administration of a MK-binding protein or conjugate or composition according to the methods of the present disclosure can be continuous or intermittent, depending, for example, on the recipient's physiological condition, whether the purpose of the administration is therapeutic or prophylactic, and other factors known to skilled practitioners. The administration of a MK-binding protein or conjugate or composition may be essentially continuous over a preselected period of time or may be in a series of spaced doses.

A variety of routes of administration are possible including, but not necessarily limited to, oral, dietary, topical, parenteral (e.g., intravenous, intraarterial, intramuscular, subcutaneous injection), inhalation (e.g., intrabronchial, intraocular, intranasal or oral inhalation, intranasal drops), depending on the bone disease, disorder, condition and/or injury to be treated. Other suitable methods of administration can also include rechargeable or biodegradable devices and slow release polymeric devices.

### Combination Therapies

In one example of a method described herein, the isolated or recombinant protein or conjugate or composition as described is administered in combination with another compound useful for promoting bone formation and/or promoting bone healing and/or increasing BMD, either as combined or additional treatment steps or as additional components of a therapeutic formulation.

For example, the other compound may be bisphosphonate, such as e.g., Alendronate (Foxamax), Risedronate (Actonel), Ibandronate (Boniva) or Zoledronic acid (Reclast or Aclasta). Alternatively, or in addition, the other compound may be a corticosteroid e.g., prednisone or cortisone. Alternatively, or in addition, the other compound may be denosumab (Prolia). Alternatively, or in addition, the other compound may be strontium ranelate (Protos). Alternatively, or in addition, the other compound may be a selective oestrogen receptor modulator (SERMS), such as raloxifene (Evista). Alternatively, or in addition, the other compound may be a drug used in hormone replacement therapy (HRT), such as oestrogen or progesterone. Alternatively, or in addition, the other compound may be teriparatide (Forteo). Alternatively, or in addition, the other compound may be a non-steroidal anti-inflammatory agent or analgesic. For example, a suitable non-steroidal anti-inflammatory agent may be ibuprofen, naproxen or a COX-1 and/or COX-2 inhibitor selected from ketoprofen, indomethacin (Indocin or Tivorbex), fenoprofen (Nalfon).

The present invention will now be described more specifically with reference to the following non-limiting Examples.

### EXAMPLES

### Example 1—Effect of Midkine Antibody on Adult Fracture Healing

In this example, the inventors evaluated the effects of subcutaneously administered anti-midkine antibody on fracture healing in adult mice.

### Methods

### Animals

All mice used in this experiment were female C57BL/6J mice provided by the University of Ulm. The mice were maintained in groups of two to four animals per cage (370 cm²) on a 14 h light and 10 h dark circadian rhythm with water and food ad libitum.

### Treatment

Briefly, nine-month-old mice were randomly divided into two groups, Group 1 (n=X) and Group 2 (n=X), and each animal received a standardized osteotomy at the midshaft of the right femur using a 0.4 mm Gigli saw (RISystem, Davos, Switzerland) stabilized using an external fixator (axial stiffness of 3.0 N/mm, RISystem). Immediately following surgery, treatment was initiated. The animals in Group 1 were treated with anti-midkine antibody, IP-10, which was administered subcutaneously at 25 mg/kg twice weekly for 3 weeks. The animals of Group 2 were treated in parallel using the vehicle phosphate-buffered saline (PBS). Animals from each of Groups 1 and Group 2 were sacrificed 4, 10, 21 or 28 days post-surgery using carbon dioxide (n=6-8 per group at each time point). Blood samples were collected from mice sacrificed at days 0, 4, 10 or 21 post-surgery. The fractured and intact femurs were removed from all mice for further analysis.

### Serum Analysis

The midkine protein serum level was determined for each animal using a human midkine enzyme-linked immunosorbent assay kit (provided by Cellmid Ltd) in accordance with the manufacturer's protocol, which was cross-reactive with murine midkine.

### Biomechanical Testing

Biomechanical testing of the intact and fractured femurs of the mice sacrificed at day 21, 23 or 28 was performed using a nondestructive 3-point-bending test as described in Röntgen et al., (2010) J. Orthop. Res., 28:1456-1462. Briefly, after removal of the fixator, a bending load (maximum 4 N) was applied to the top of the carnio-lateral callus side. The flexural rigidity of the bones was calculated using the slope of the load-deflection curve. The relative flexural rigidity of the fractured femur was calculated as the ratio between the fractured and intact femur of the same mouse.

### Micro-Computed Tomography (μCT)

Femurs from the mice were analyzed using a μCT scanning device (Skyscan 1172, Kontich, Belgium) operating at a voxel resolution of 8 μm (50 kV, 200 mA). Four volumes of interest (VOIs) were determined for μCT analysis: VOI 1 covered the callus between the fractured cortices and VOI 2 covered the periosteal callus between the two inner pin holes. VOI 3 (intact cortical bone) covered the area from 80 μm proximal to 80 μm distal from the middle of the diaphysis of the intact femur. The starting point for VOI 4 (intact trabecular bone) was 200 μm proximal to the metaphyseal growth plate of the intact femur and the endpoint was 280 μm proximal from the starting point. VOI 5 covered the trabecular part of the second lumbar vertebral body. Bone mineral density (BMD) was assessed using two phantoms with a defined hydroxyapatite density (250 mg/cm³ and 750 mg/cm³) within each scan. BMD of the fracture callus was evaluated without a threshold, whereas BMD of the cortical bone was determined using a global threshold of 642 mg hydroxyapatite/cm³ according to the methods described in Morgan et al., (2009) Bone, 44(2):225-244. BMD of the trabecular bone was evaluated using a global threshold of 395 mg hydroxyapatite/cm³ as described in Wehrle et al., (2014) J. Orthop. Res., 32(8):1006-1013 and O'Neill et al, (2012) Bone, 50:1357-67. Analysis of the bone parameters was performed using Skyscan software (NRecon, DataViewer, CTAn) in accordance with the guidelines set forth by the American Society for Bone and Mineral Research (ASBMR) for μCT analysis as described in Bouxsein et al., (2010) J. Bone Miner. Res., 25(7):1468-1486.

### Histomorphometry of Undecalcified Femora

The amounts of bone, cartilage and fibrous tissue in the whole callus between the two inner pin holes were determined using undecalcified histological sections of fractured femurs explanted at day 21 and 28. Briefly, femurs were fixed in 4% formalin, dehydrated in ascending ethanol series and embedded in methyl methacrylate. Cross-sections of 7 μm were prepared and stained using Giemsa for histomorphometric analysis. The callus tissue was examined using light microscopy (DMI6000 B, Leica, Heerbrugg, Switzerland). The amounts of bone, cartilage, and fibrous tissue were determined using image-analysis software (Leica MMAF 1.4.0 Imaging System, Leica). To identify osteoblasts and osteoblast surface, cross-sections of 7 μm were prepared and stained using Toluidine Blue and analyzed under 400-fold magnification. Tartrate-resistant acid phosphatase staining was used to identify osteoclast numbers as described in Heilmann et al., (2013) PLoS One, 8(12):e84232.

### Histomorphometry of Decalcified Femora and Immunohistochemistry

Femurs of mice sacrificed 4, 10 or 21 days post-surgery were fixed in 4% formalin, decalcified using 20% ethylenediaminetetraacetic acid (pH 7.2-7.4) for 10-12 days and embedded in paraffin after dehydration in ascending ethanol series. Longitudinal cross-sections of 7 μm thickness were prepared and stained using Safranin O for tissue quantification. Immunohistochemical staining of midkine and beta-catenin was performed using the following antibodies: polyclonal goat anti-mouse Mdk antibody (sc-1398, Santa Cruz Biotechnology, Dallas, USA), polyclonal rabbit anti-mouse beta-catenin antibody (AB 19022, EMD Millipore Corporation, Merck, Darmstadt, Germany), HRP-conjugated Streptavidin (Zytomed Systems, Berlin, Germany), donkey anti-goat IgG F(ab′)2 biotin-conjugated (sc-3854, Santa Cruz Biotechnology) and goat anti-mouse IgG (Invitrogen, ThermoFisher Scientific, Waltham, USA). Species-specific non-targeting immunoglobulins were used as isotype controls. β-Amino-9-ethylcarbazol (Zytomed Systems) was used as the chromogen and the sections were counterstained using hematoxylin (Waldeck, Münster, Germany). Quantification of the positively stained regions for beta-catenin was performed using the image analysis software Adobe Photoshop CS4 (Adobe, Dublin, Ireland). The color gamut of positive staining was determined with the color picker tool and a tolerance of 40. The positively stained pixels were counted in the histogram and calculated against all pixels of the image to determine the percentage of positively stained area.

### Statistical Analysis

Statistical analysis for the experiments using 9-month-old mice was performed using the non-parametric Mann-Whitney-U test with SPSS software (SPSS Inc., Chicago, USA). All results are presented as box plots with median, first and third quartiles and maximum and minimum values. Outliers (value is less than the first quartile minus 1.5 times the interquartile range or greater than the third quartile plus 1.5 times the interquartile range) were marked as small circles. Values of p<0.05 were considered to be statistically significant.

### Results

Treatment with Anti-Midkine Antibody Increased the Mechanical Competence and Bone Formation of Adult Mice Fracture Callus

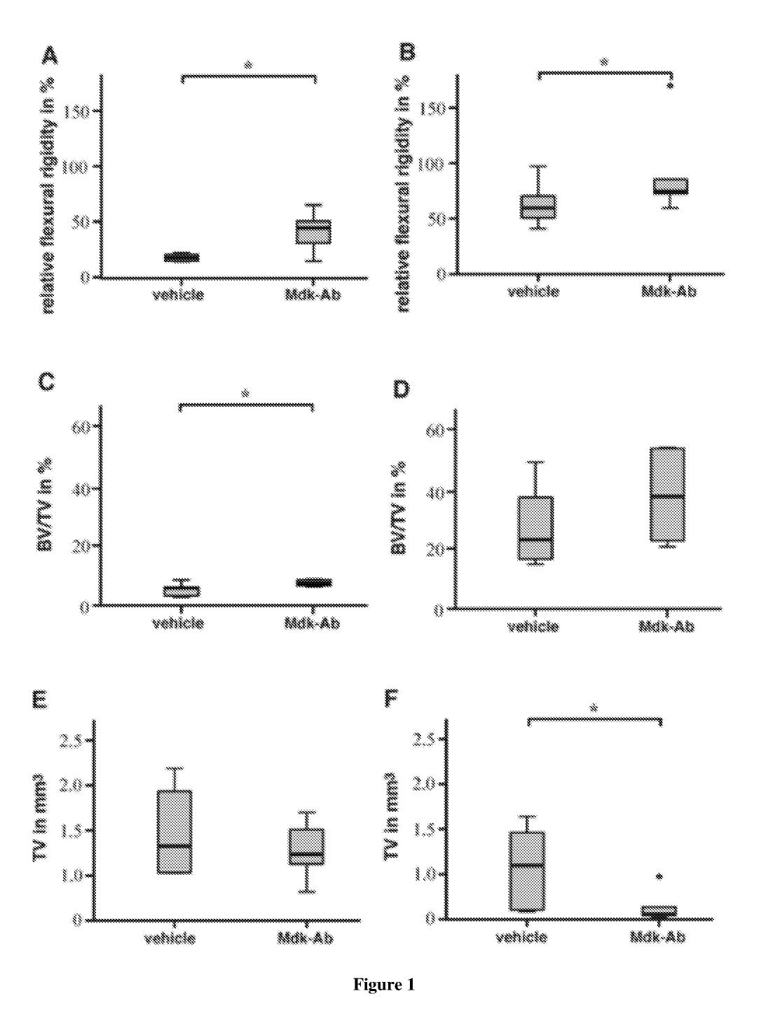

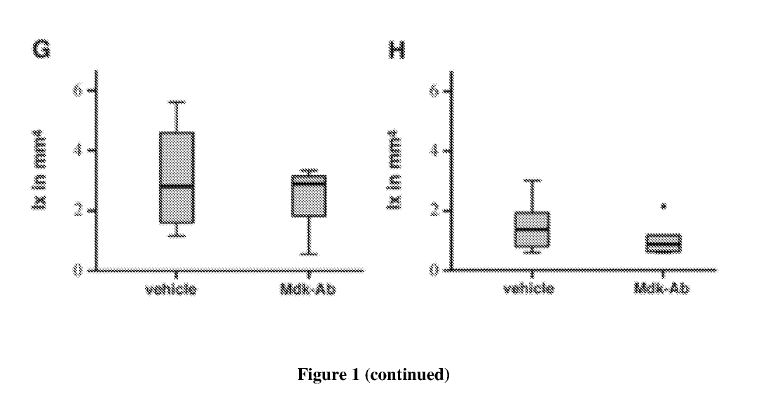

The biomechanical testing demonstrated that treatment with the anti-midkine antibody significantly increased the relative flexural rigidity of the fractured femurs of 9-month-old mice after both 21 and 28 days of healing compared to the control group (FIGS. 1, A and B). Furthermore, μCT analysis of the fracture callus revealed an enhanced bone volume/tissue volume (BV/TV) after both 21 and 28 days of healing compared to the control group (FIGS. 1, C and D). Callus size and geometry were unaffected at an intermediate time point of fracture healing i.e., day 21 (FIGS. 1, E and G), whereas the TV was significantly lower in animals treated with anti-midkine antibody after 28 days of healing (FIG. 1F), indicating an earlier remodeling phase in these mice.

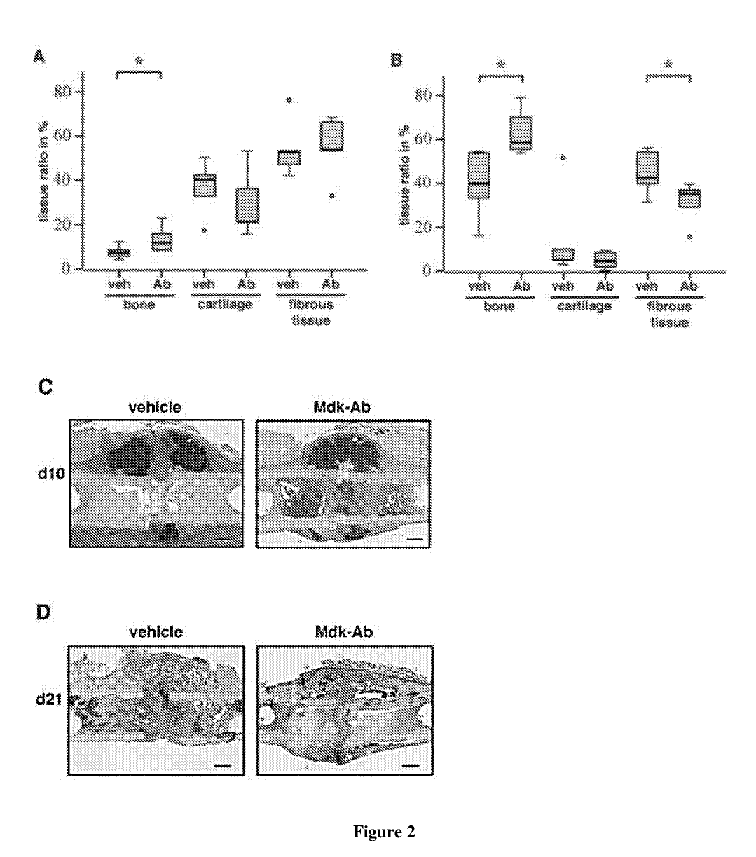

Histomorphometric analysis of the fracture callus tissue composition showed that animals treated with anti-midkine antibody displayed a significantly increased amount of newly formed bone after both 10 and 21 days (FIG. 2). No significant differences were observed in the fracture callus tissue composition at the late stage of fracture healing (day 28, data not shown).

Midkine Protein Expression was Differentially Affected by Treatment with Anti-Midkine Antibody in Fracture Callus Osteoblasts and Chondrocytes

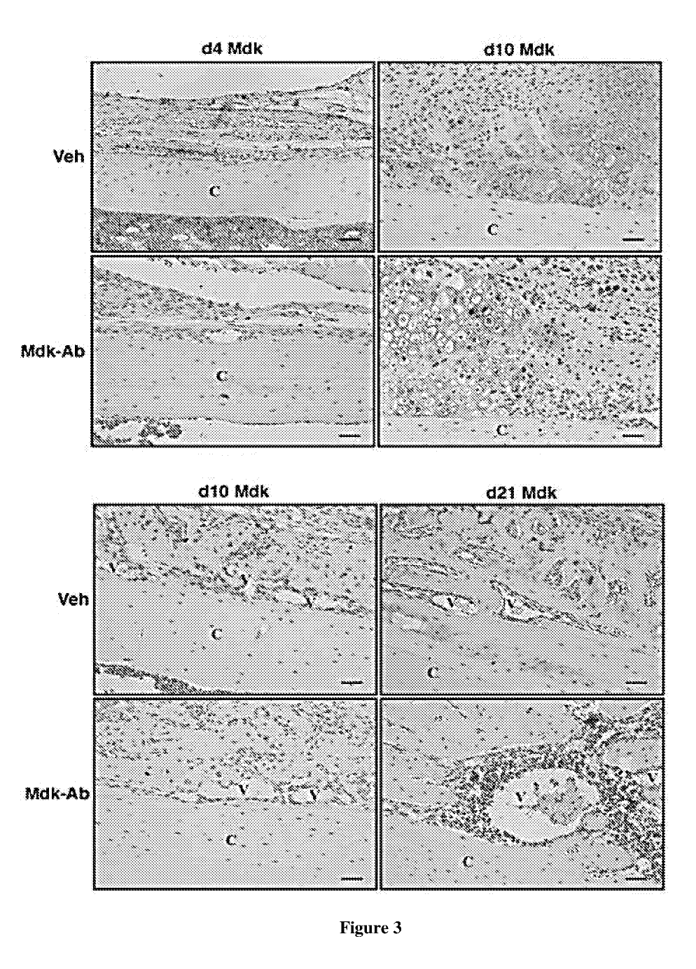

Since midkine protein has been shown to be expressed in several cell types during fracture healing, the inventors also analyzed the impact of treatment with anti-midkine antibody on midkine expression (FIG. 3).

At day 4 after surgery, midkine protein was shown to be expressed in the periosteal region of the fracture callus, which was attenuated by treatment with anti-midkine antibody. Midkine protein expression peaked 10 days after surgery in vehicle-treated mice. The protein was located intracellularly in proliferating and hypertrophic chondrocytes and extracellularly in areas of new bone formation. Midkine protein expression was clearly greater around the vessels in the periosteal fracture callus, which was attenuated by treatment with anti-midkine antibody. Midkine protein was detected intracellularly in chondrocytes in both vehicle- and antibody-treated mice. At day 21, expression of midkine protein was only observed in scattered chondrocytes and was very low in areas of new bone formation. There were no differences between vehicle- and antibody-treated mice at this time point.

### Osteotomy Increased Midkine Protein Serum Levels

Because significant differences in midkine expression in areas of neovascularization in the fracture callus were identified between animals in Group 1 and Group 2, the inventors investigated midkine serum levels before and after the osteotomy.

Untreated mice at day 0 displayed only low levels of midkine protein in serum. In control animals, serum midkine protein levels were more than doubled at day 4 (58-68 pg/ml) and peaked at day 10 (78-91 pg/ml), returning to pre-operation levels by day 21 (Table 1).

TABLE 1
Midkine (Mdk) serum levels during fracture
healing in 9-month-old mice.

| | | |
|---|---|---|
| days after operation | vehicle | Mdk-Ab |

| | |
|---|---|
| d 0 | 7.2 ± 12.3 |

| | | |
|---|---|---|
| d 4 | 63.8 ± 4.9# | 8.9 ± 15.4 |
| d 10 | 86.4 ± 7.3# | 29.7 ± 31.7* |
| d 21 | 9.6 ± 16.7 | n.d. |

#p < 0.05 for effect of fracture
*p < 0.05 for effect of Mdk-Ab
n.d.: not detectable; d 0: pre-operation value. (n = 3-7 per group presented as mean ± standard deviation.) Mann-Whitney-U test.

Treatment with the anti-midkine antibody slightly decreased the level of midkine protein in serum at day 4 and significantly at day 10 compared to vehicle-treated mice.

Treatment with Anti-Midkine Antibody Increased Beta-Catenin Expression and Osteoblast Activity

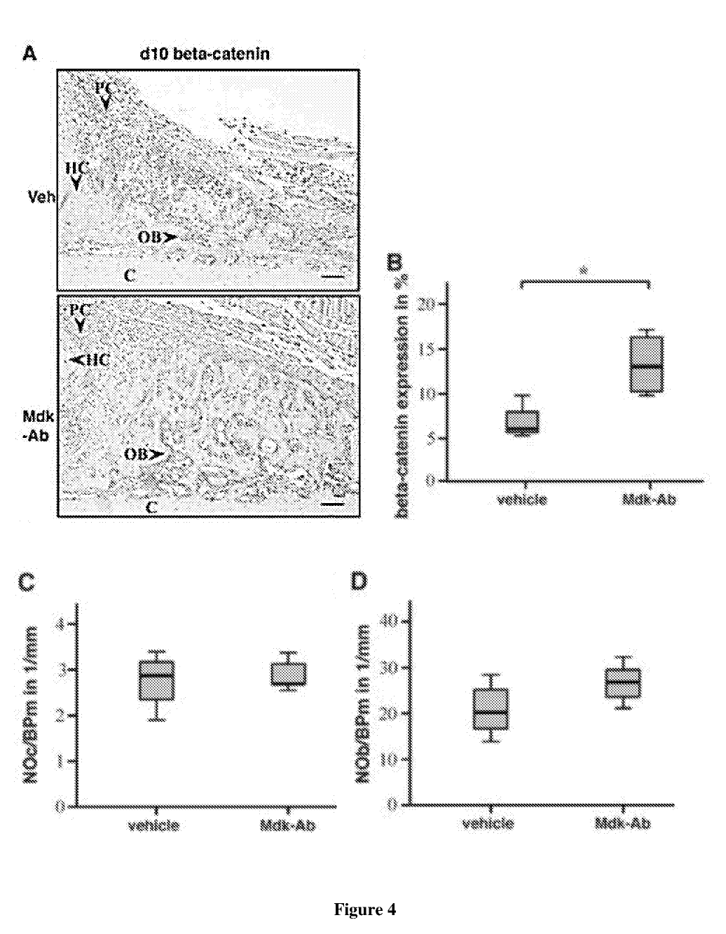

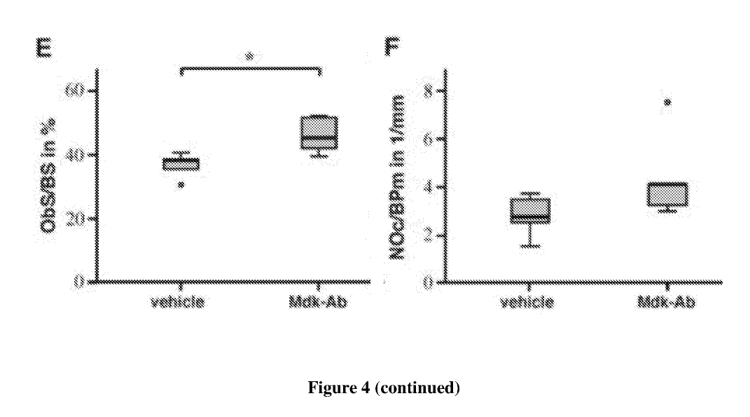

As midkine has previously been shown to influence Wnt/beta-catenin signaling in osteoblasts (Liedert et al., (2011) Bone, 48:945-951), the inventors evaluated the effects of treatment with anti-midkine antibody on beta-catenin signaling during fracture healing. Proliferating chondrocytes and osteoblasts were immunohistochemically stained for beta-catenin at day 10 after fracture (FIG. 4A), whereas hypertrophic chondrocytes were beta-catenin negative. At day 10, the percentage of beta-catenin positive area was significantly increased in the fracture calli of the animals treated with anti-midkine antibody (FIG. 4B).

Because midkine-deficient mice have previously been shown to display no differences in the number of osteoclasts or osteoblasts in the fracture callus (Haffner-Luntzer et al., (201) PLoS One, 9(12):e1116282), the number of osteoclasts or osteoblasts was analysed in the fracture callus of animals in Group 1 and Group 2 to determine the effect of treatment with the anti-midkine antibody. At day 21, the number of osteoclasts was similar in both groups (FIG. 4C) while the number of osteoblasts was only slightly increased after antibody treatment (p=0.151; FIG. 4D). In contrast, the osteoblast surface was significantly increased after treatment with the anti-midkine antibody (FIG. 4E), indicating enhanced osteoblast activity. At day 28, the number of osteoclasts slightly increased in the antibody-treated mice (p=0.082; FIG. 4F), indicating more rapid callus remodeling.

### Example 2—Effect of Midkine Antibody on Fracture Healing in Young and Osteoporotic Mice

Since osteoporotic patients display reduced bone healing capacity and delayed fracture healing, the inventors evaluated the effect of treatment with anti-midkine antibody on fracture healing in young and osteoporotic mice.

### Methods

### Animals

All mice used in this experiment were female C57BL/6J mice provided by the University of Ulm. The mice were maintained in groups of two to four animals per cage (370 cm²) on a 14 h light and 10 h dark circadian rhythm with water and phytoestrogen-free diet ad libitum for the entirety of the experiment.

### Treatment

Briefly, three-month-old mice were randomly divided into two groups that underwent a bilateral sham-operation or ovariectomy (OVX), respectively. The sham operation and OVX were performed as described in Wehrle et al., (2014) J. Orthop. Res., 32(8):1006-1013. Eight weeks after receiving the sham operation or OVX, animals received a standardized osteotomy at the midshaft of the right femur using a 0.4 mm Gigli saw (RISystem, Davos, Switzerland) stabilized using an external fixator (axial stiffness of 3.0 N/mm, RISystem). Immediately following the osteotomy, treatment was initiated. During treatment, half the animals in the sham-operated group and half the animals in the OVX group received anti-midkine antibody, IP-10, which was administered subcutaneously at 25 mg/kg twice weekly for 3 weeks. The remaining animals in the sham-operated and OVX groups were treated in parallel using the vehicle phosphate-buffered saline (PBS). Animals from each of the four groups were sacrificed on days 0, 3, 10 and 23 post-osteotomy using carbon dioxide (n=6-7 per group at each time point). Blood samples were collected from mice sacrificed at each time point. The fractured and intact femurs, as well as the lumber vertebral bodies were removed from all mice for further analysis.

### Serum Analysis

The midkine protein serum level was determined for each animal using a human midkine enzyme-linked immunosorbent assay kit (provided by Cellmid Ltd) in accordance with the manufacturer's protocol, which was cross-reactive with murine midkine.

### Biomechanical Testing

Biomechanical testing of the intact and fractured femurs of the mice sacrificed at day 23 was performed using a nondestructive β-point-bending test as described in Röntgen et al., (2010) J. Orthop. Res., 28:1456-1462. Briefly, after removal of the fixator, a bending load (maximum 4 N) was applied to the top of the carnio-lateral callus side. The flexural rigidity of the bones was calculated using the slope of the load-deflection curve. The relative flexural rigidity of the fractured femur was calculated as the ratio between the fractured and intact femur of the same mouse.

### Micro-Computed Tomography (μCT)

Femurs and vertebral bodies from the mice were analyzed using a μCT scanning device (Skyscan 1172, Kontich, Belgium) operating at a voxel resolution of 8 μm (50 kV, 200 mA). Four volumes of interest (VOIs) were determined for μCT analysis: VOI 1 covered the callus between the fractured cortices and VOI 2 covered the periosteal callus between the two inner pin holes. VOI 3 (intact cortical bone) covered the area from 80 μm proximal to 80 μm distal from the middle of the diaphysis of the intact femur. The starting point for VOI 4 (intact trabecular bone) was 200 μm proximal to the metaphyseal growth plate of the intact femur and the endpoint was 280 μm proximal from the starting point. VOI 5 covered the trabecular part of the second lumbar vertebral body. Bone mineral density (BMD) was assessed using two phantoms with a defined hydroxyapatite density (250 mg/cm³ and 750 mg/cm³) within each scan. BMD of the fracture callus was evaluated without a threshold, whereas BMD of the cortical bone was determined using a global threshold of 642 mg hydroxyapatite/cm³ according to the methods described in Morgan et al., (2009) Bone, 44(2):225-244. BMD of the trabecular bone was evaluated using a global threshold of 395 mg hydroxyapatite/cm³ as described in Wehrle et al., (2014) J. Orthop. Res., 32(8):1006-1013 and O'Neill et al, (2012) Bone 50:1357-67. Analysis of the bone parameters was performed using Skyscan software (NRecon, DataViewer, CTAn) in accordance with the guidelines set forth by the American Society for Bone and Mineral Research (ASBMR) for μCT analysis as described in Bouxsein et al., (2010) J. Bone Miner. Res., 25(7):1468-1486.

### Histomorphometry of Undecalcified Femora

The amounts of bone, cartilage and fibrous tissue in the whole callus between the two inner pin holes were determined using undecalcified histological sections of fractured femurs explanted at day 23 post-osteotomy. Briefly, femurs were fixed in 4% formalin, dehydrated in ascending ethanol series and embedded in methyl methacrylate. Cross-sections of 7 μm were prepared and stained using Giemsa for histomorphometric analysis. The callus tissue was examined using light microscopy (DMI6000 B, Leica, Heerbrugg, Switzerland). The amounts of bone, cartilage, and fibrous tissue were determined using image-analysis software (Leica MMAF 1.4.0 Imaging System, Leica). To identify osteoblasts and osteoblast surface, cross-sections of 7 μm were prepared and stained using Toluidine Blue and analyzed under 400-fold magnification. Tartrate-resistant acid phosphatase staining was used to identify osteoclast numbers as described in Heilmann et al., (2013) PLoS One, 8(12):e84232.

### Histomorphometry of Decalcified Femora and Immunohistochemistry

Femurs of mice sacrificed 3 or 10 days post-osteotomy were fixed in 4% formalin, decalcified using 20% ethylenediaminetetraacetic acid (pH 7.2-7.4) for 10-12 days and embedded in paraffin after dehydration in ascending ethanol series. Longitudinal cross-sections of 7 μm thickness were prepared and stained using Safranin O for tissue quantification. Immunohistochemical staining of midkine and beta-catenin was performed using the following antibodies: polyclonal goat anti-mouse midkine antibody (sc-1398, Santa Cruz Biotechnology, Dallas, USA), polyclonal rabbit anti-mouse beta-catenin antibody (AB 19022, EMD Millipore Corporation, Merck, Darmstadt, Germany), HRP-conjugated Streptavidin (Zytomed Systems, Berlin, Germany), donkey anti-goat IgG F(ab′)2 biotin-conjugated (sc-3854, Santa Cruz Biotechnology) and goat anti-mouse IgG (Invitrogen, ThermoFisher Scientific, Waltham, USA). Species-specific non-targeting immunoglobulins were used as isotype controls. β-Amino-9-ethylcarbazol (Zytomed Systems) was used as the chromogen and the sections were counterstained using hematoxylin (Waldeck, Münster, Germany). Quantification of the positively stained regions for beta-catenin was performed using the image analysis software Adobe Photoshop CS4 (Adobe, Dublin, Ireland). The color gamut of positive staining was determined with the color picker tool and a tolerance of 40. The positively stained pixels were counted in the histogram and calculated against all pixels of the image to determine the percentage of positively stained area.

### Statistical Analysis

Statistical analysis for the experiments using sham-operated/OVX mice were analyzed for significance using the Kruskal-Wallis test with Dunn's post hoc test. All results are presented as box plots with median, first and third quartiles and maximum and minimum values. Outliers (value is less than the first quartile minus 1.5 times the interquartile range or greater than the third quartile plus 1.5 times the interquartile range) were marked as small circles. Values of p<0.05 were considered to be statistically significant.

### Results

Treatment with Anti-Midkine Antibody Accelerated Osteoporotic Fracture Healing

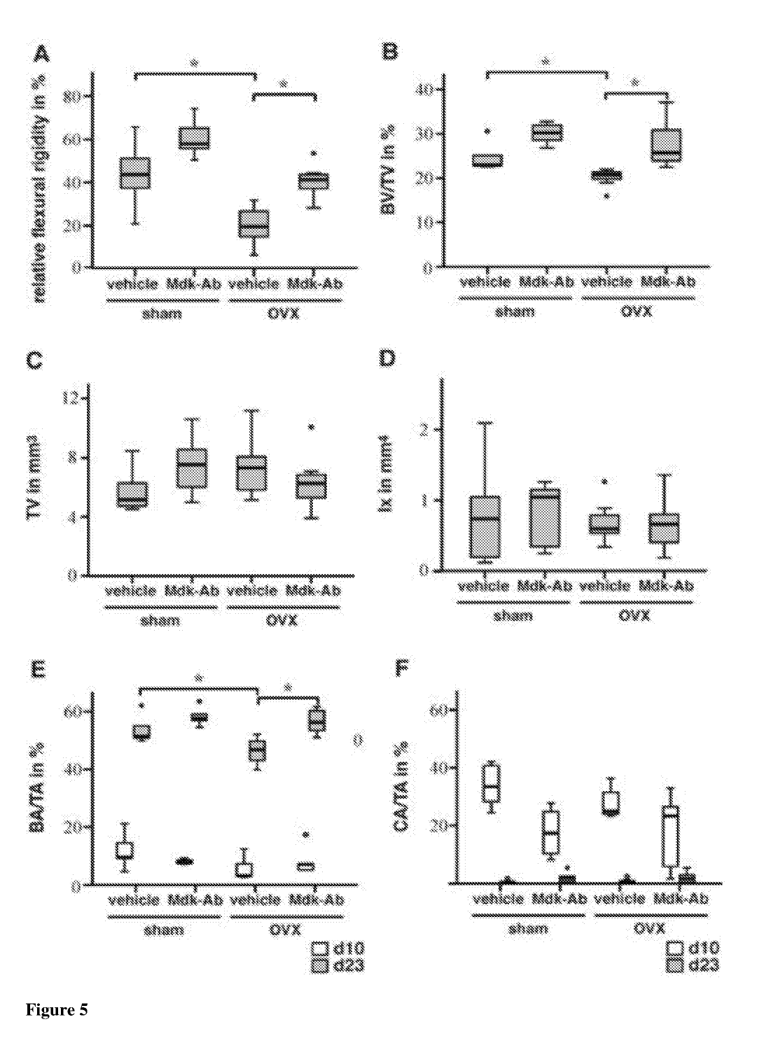

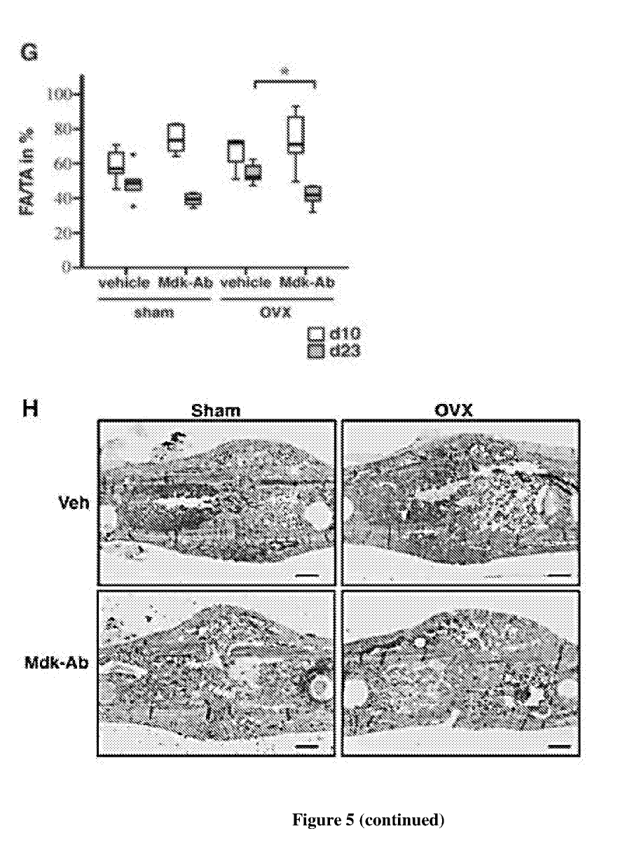

Biomechanical testing confirmed that OVX significantly decreased the relative flexural rigidity in the vehicle-treated mice. Treatment with the anti-midkine antibody slightly increased the relative flexural rigidity in the sham-operated group and significantly in the OVX group (FIG. 5A). μCT analysis of the fracture calli demonstrated decreased BV/TV after OVX and an enhanced BV/TV after antibody treatment (FIG. 5B). Callus size and geometry were unaffected by OVX or midkine-Ab treatment (FIGS. 5, C and D). Histomorphometry analysis of the fracture callus tissue composition showed no significant differences between the treatment groups at day 10 after surgery (FIG. 5, E-G). However, at day 23, the fracture callus bone content was significantly decreased after OVX and significantly reversed on treatment with the anti-midkine antibody (FIGS. 5, E and H). Fibrous tissue content was significantly decreased in the OVX mice after treatment with the anti-midkine antibody (FIGS. 5, G and H).

Treatment with Anti-Midkine Antibody Decreased Mdk Serum Levels in Osteoporotic Mice

Midkine protein serum levels 0, 3, 10 and 23 days after fracture are presented in Table 2. As is apparent, none of the mice had a detectable circulating midkine protein at day 0. Fracture increased the midkine protein serum levels in both sham-operated and OVX mice after 3 days. However, the midkine protein serum level remained elevated only in OVX mice until day 23. At day 10, treatment with anti-midkine antibody resulted in significantly decreased midkine protein serum levels in OVX animals.

TABLE 2
Midkine (Mdk) serum levels during fracture healing in 3-month-
old sham-operated and ovariectomized (OVX) mice; n.d.: not detectable.
d 0: pre-operation value. (n = 5-7 per group presented
as the mean ± standard deviation.) Kruskal-Wallis test.

| | treatment | | | |
|---|---|---|---|---|
| days after | sham | | OVX | |
| operation | vehicle | Mdk-Ab | vehicle | Mdk-Ab |
| d 0 | n.d. | | n.d. | |
| d 3 | 38.6 ± 44.6 | 15.1 ± 33.9 | 67.9 ± 45.7 | 37.8 ± 43.6 |
| d 10 | n.d. | n.d. | 61.7 ± 43.6^{#} | n.d. * |
| d 23 | n.d. | n.d. | 31.0 ± 28.2 | n.d. |

^{#}p < 0.05 for effect of OVX
* p < 0.05 for effect of Mdk-Ab

Treatment with Anti-Midkine Antibody Abolished OVX-Induced Decreased Beta-Catenin Expression

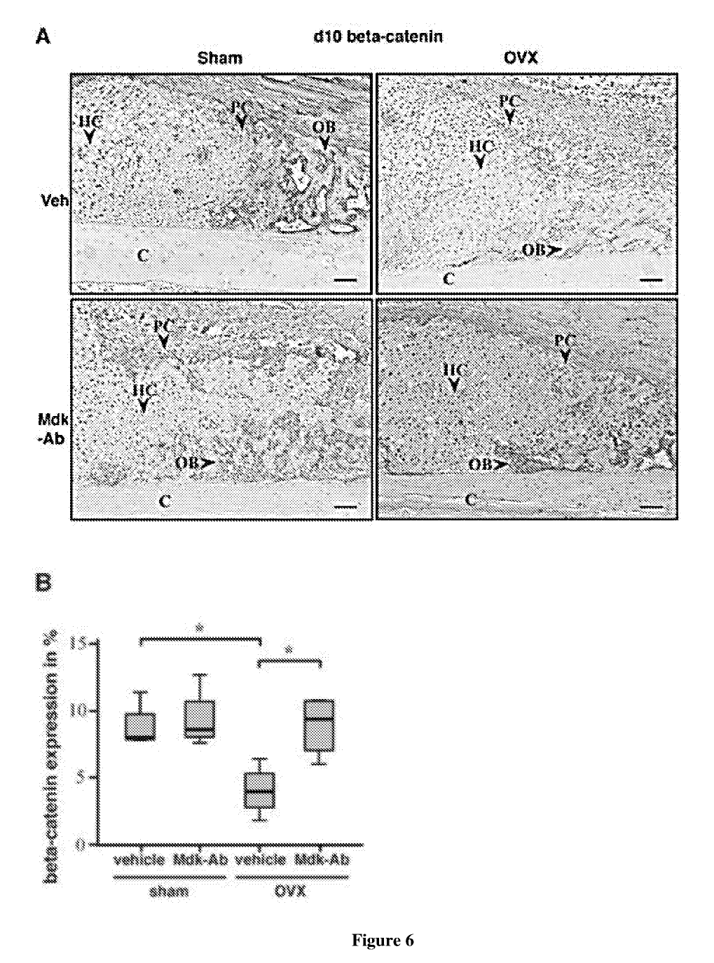

OVX significantly decreased the beta-catenin positive area in the fracture callus of young mice compared to the sham-operated animals at day 10 (FIG. 6). Treatment with anti-midkine antibody did not alter the beta-catenin expression in sham-operated mice, whereas there was a significant increase in the beta-catenin positive area percentage in OVX mice.

Treatment with Anti-Midkine Antibody Enhanced the Bone Content in the Intact Femur and the Vertebral Bodies of Osteoporotic Mice

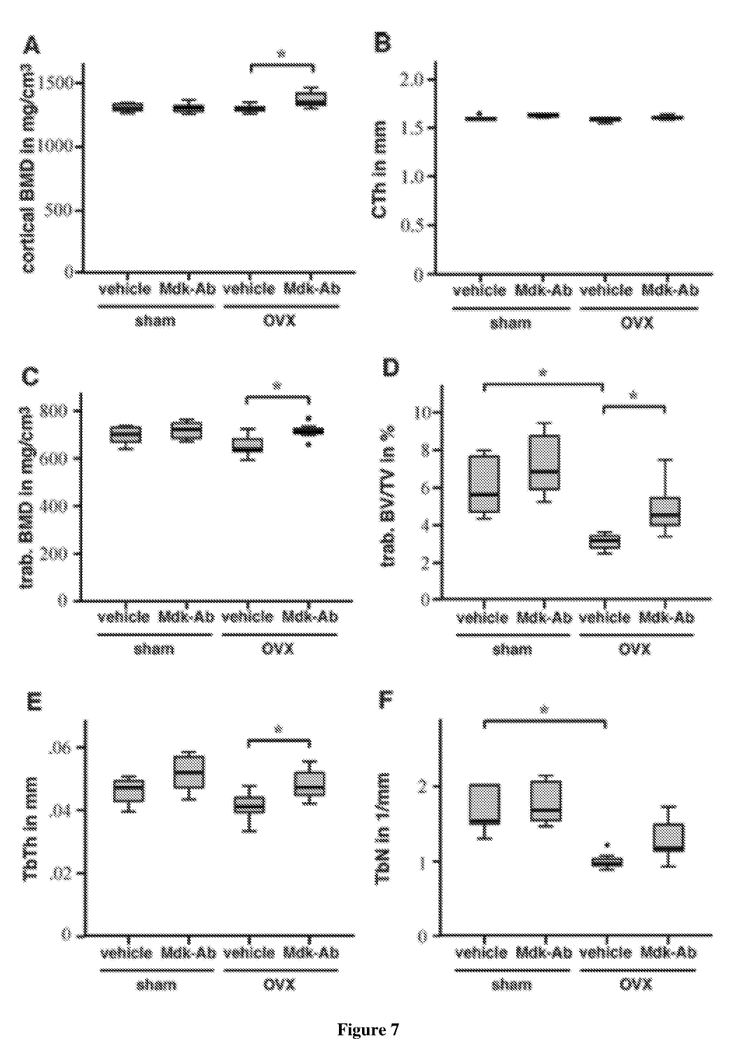

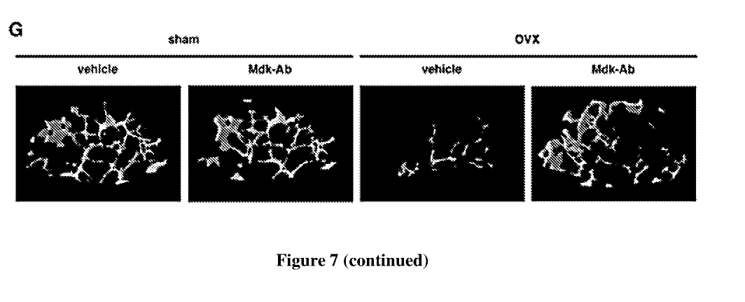

Because the bone content in the callus of OVX mice was significantly enhanced by treatment with anti-midkine antibody, the effect on the intact bone was also investigated. Antibody treatment increased the cortical BMD in the intact femur of OVX animals (FIG. 7 A), whereas cortical thickness was unaffected in all groups (FIG. 7 B). Evaluation of the trabecular region of the intact femur confirmed an osteoporotic phenotype in OVX mice; trabecular BV/TV and number were significantly decreased in vehicle-treated mice. Treatment with anti-midkine antibody significantly increased trabecular BMD, BV/TV and thickness in OVX mice (FIG. 7, C-G). Bone parameters in sham-operated mice were only slightly increased after antibody treatment.

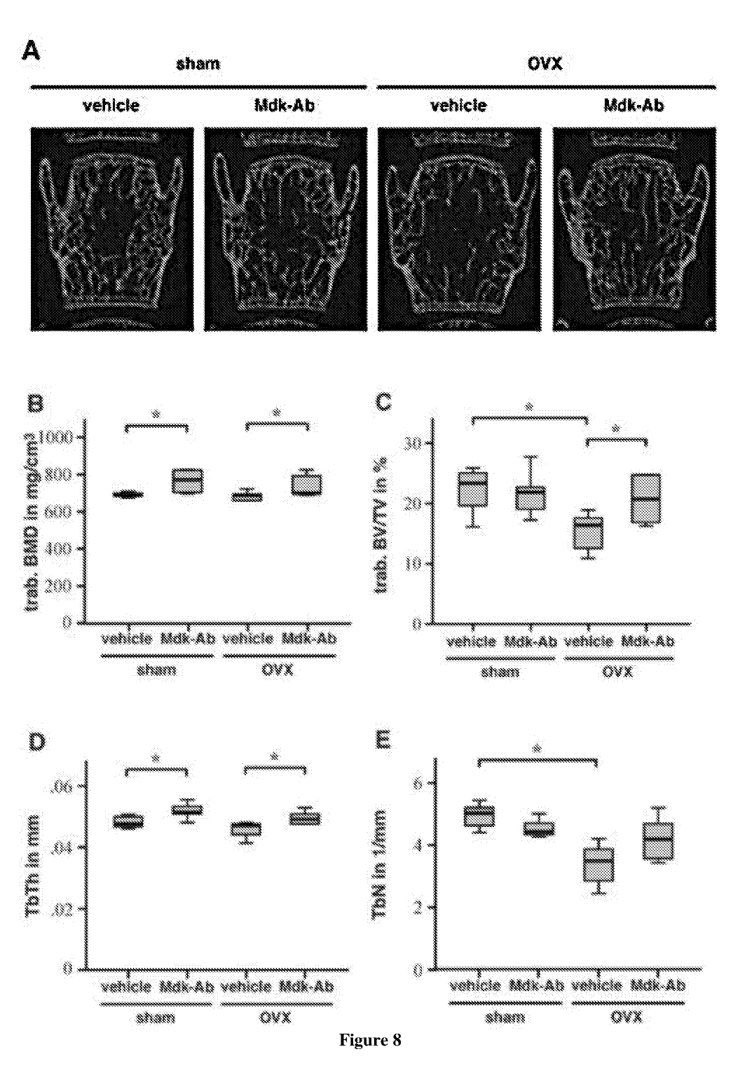

In agreement with the femur, OVX also led to a decreased trabecular BV/TV and number in the vertebral bodies of vehicle-treated mice (FIG. 8). Treatment with anti-midkine antibody significantly increased the vertebral trabecular BMD, BV/TV and thickness in the OVX animals (FIG. 8, B-D). The antibody treatment had a great impact on the trabecular bone of the spine than of the femur in sham-operated mice; trabecular BMD and thickness were significantly improved by treatment with anti-midkine antibody, whereas trabecular BV/TV and number remained unchanged in the sham-operated animals (FIGS. 8, C and E).

### Example 3—Treatment of Cells with Anti-Midkine Antibody Diminished the Negative Influence of Midkine on Beta-Catenin Signaling In Vitro

Based on the finding that osteoblast activity is enhanced after treatment with anti-midkine antibody during fracture healing in mice, the inventors sought to determine the effects of treatment with midkine protein or anti-midkine antibody in MC3T3-E1 cells, ATDC5 cells, C57BL/6 primary osteoblasts and C57BL/6 mesenchymal stem cells in vitro.

### Methods

### Cell Culture Conditions

Preosteoblastic MC3T3-E1 cells were obtained from Sigma-Aldrich, Germany and cultured in α-minimum essential medium (Gibco, ThermoFisher Scientific) containing 10% fetal calf serum (FCS) (PAA Laboratories, Cölbe, Germany), 1% penicillin/streptomycin (Gibco) and 1% L-glutamine (Biochrom, Merck). Osteogenic differentiation was induced by adding 10 mM β-glycerophosphate and 0.2 mM ascorbate-2-phosphate (both Sigma-Aldrich) to the culture medium. Cells were seeded in 6- or 24-well plates at 20,000 cells/cm² for proliferation and differentiation experiments, respectively. In cyclic stretching experiments, cells were plated in FCS-coated silicon dishes at 200,000 cells per dish.

ATDC5 chondroprogenitor cells were obtained from Sigma-Aldrich and cultured in a 1:1 mixture of Dulbecco's Modified Eagle's Medium and Ham's F12 medium (Gibco, ThermoFisher Scientific) containing 5% FCS (PAA Laboratories), 1% penicillin/streptomycin (Gibco, ThermoFisher Scientific), 1% L-glutamine (Biochrom, Merck), 10 μg/ml human transferrin and 3×10⁻⁸ M sodium selenite (both Sigma-Aldrich) as described in Shukunami et al., (1996) J. Cell Biol., 133:457-468. Cells were seeded in 6-well plates at 10,000 cells/cm². Chondrogenic differentiation was induced by supplementing normal culture medium with 10 μg/ml human insulin (Sigma-Aldrich) and 5 ng/ml human transforming growth factor beta1 (R&D Systems, Minneapolis, USA).

Murine primary osteoblasts were obtained from β-4-day-old WT C57BL/6 mice and cultured according to the conditions described in Schmidt et al., (2005) J. Cell Biol., 168:899-910. Primary osteoblasts were seeded in FCS-coated silicon dishes at 200,000 cells per dish, differentiated for 21 days by adding 10 mM β-glycerophosphate and 0.2 mM ascorbate-2-phosphate to the culture medium and exposed to cyclic stretching.

Bone marrow-derived (mMSCs) were isolated by flushing the long bones of 6-week-old C57BL/6 mice, selected via plastic adherence and cultured as described in Huebner et al., (2006) J. Bone Miner. Res. 21:1924-1934. Cells were seeded at 7,500 cells per well in 24-well plates and differentiated for 10 days by adding 10 mM β-glycerophosphate and 0.2 mM ascorbate-2-phosphate to the culture medium. All experiments were performed three times in duplicate or triplicate.

In each case, recombinant midkine protein was added to the culture medium of designated cell cultures at 100 ng/ml (Dianova, Hamburg, Germany) and anti-midkine antibody at 2 p g/ml.

All experiments were performed three times in duplicate.

### 3-(4,5-Dimethylthiazol-2-yl)-2,5-diphenyltetrazolium Bromide (MTT) Assay

The proliferation of MC3T3-E1 cells and murine MSCs was determined using the MTT assay. Briefly, cells were seeded in 96-well plates at 3,000 cells per well for 24 hours, then 10 μl of 12 mM MTT, 100 ng/ml midkine protein and/or the anti-midkine antibody (2 μg/ml) were added to the designated wells and incubated for 6 hours. Supernatant was removed and 150 μl dimethylsulfoxide (DMSO) was added. After 10 minutes shaking, the absorbance was determined at 490 nm and the proliferation rate was calculated compared to the untreated cells.

### Mechanical Loading In Vitro

Mechanical loading of MC3T3-E1 cells and primary osteoblasts was performed at day 14 or 21 of differentiation by homogenous cyclic stretching as described in Kaspar et al., (2000) J. Biomech. 33:45-51. A sinusoidal strain of 2% and a frequency of 1 Hz were applied for 30 minutes. Recombinant midkine protein (100 ng/ml) and/or an anti-midkine antibody (2 μg/ml) were added 30 minutes prior to mechanical loading. Control dishes were prepared in parallel without loading.

### Real Time RT-PCR

Cells were lysed in RLT buffer (Qiagen, Hilden, Germany) containing 10 μl/ml β-mercaptoethanol (Sigma-Aldrich). Lysates were homogenized using QIAshredder columns and total RNA was isolated using the RNeasy Mini kit (both Qiagen), whereas DNA was digested using the RNase-free DNase kit (Qiagen). All steps were conducted according to the manufacturer's protocols. 1 μg of total RNA was reverse transcribed into cDNA using the Omniscript RT Kit (Qiagen) in a total volume of 20 μl according to the manufacturer's instructions. Quantitative PCR was performed using Brilliant Sybr Green QPCR Master Mix Kit (Stratagene, Amsterdam, Netherlands) according to the manufacturer's protocol in a total volume of 25 μl. Glyceraldehyde 3-phosphate dehydrogenase was used as housekeeping gene (F: 5′-ACC CAG AAG ACT GTG GAT GG-3′ and R: 5′-GGA TGC AGG GAT GAT GTT CT-3). Osteogenic cell differentiation was analyzed using specific primers for alkaline phosphatase (Alpl; F: 5′-GCT GAT CAT TCC CAC GTT TT-3′ and R: 5′-GAG CCA GAC CAA AGA TGG AG-3′). Relative gene expression was calculated using the delta-deltaCT method with PCR efficiency correction using LinRegPCR software as described in Ramakers et al., (2003) Neuroscience Letters, 339:62-66.

### Western Blot Analysis

10 μg of cellular lysate protein was resolved using sodium dodecyl sulfate (SDS)-polyacrylamide gel electrophoresis and transferred to a nitrocellulose membrane (BioRad, Hercules, USA). The membranes were incubated with antibody to alpha-tubulin, cFOS, beta-catenin, phospho-beta-catenin (Ser33/37/Thr41) (all Cell Signaling, Merck Millipore, Darmstadt, Germany), alkaline phosphatase (R&D Systems), LRP-1 (Abcam), LRP-6, phospho-LRP-6 (both Cell Signaling) or Mdk (Santa Cruz Biotechnologies) overnight at 4° C., respectively. To visualize the proteins, the membranes were incubated with horseradish peroxidase-conjugated secondary antibodies and developed in SuperSignal West Pico Chemiluminescent Substrate (Perbio Science, ThermoFisher Scientific). The protein bands were visualized using the Fusion Molecular Imaging System (Vilber Lourmat, Eberhardzell, Germany).

### Co-Immunoprecipitation

Cells were differentiated for 5 days in 6-well plates and incubated with midkine protein for 1 hour in ice-cold PBS. Cross-linker solution (10 mM DSP) was added for 30 minutes at room temperature. Cells were incubated with stop solution (1M Tris) for 15 minutes, washed twice, and lysed using IP-lysis buffer (Pierce, ThermoFisher Scientific). Cell debris were removed by centrifugation at 12,000 g for 10 minutes at 4° C. Protein A-sepharose beads coupled with either goat IgG or goat anti-midkine antibody (Santa Cruz Biotechnology) were added to the solution and incubated overnight at 4° C. Complexes were centrifuged at 12,000 g for 1 min and washed with lysis buffer. Protein complexes were lysed from the beads by incubating in SDS sample buffer (125 mM Tris/HCl+8.5% glycerine+1% SDS+0.1% DTT) for 5 minutes at 96° C. and for 30 minutes at 37° C. Co-immunoprecipitated proteins were visualized by western blotting.

### Results

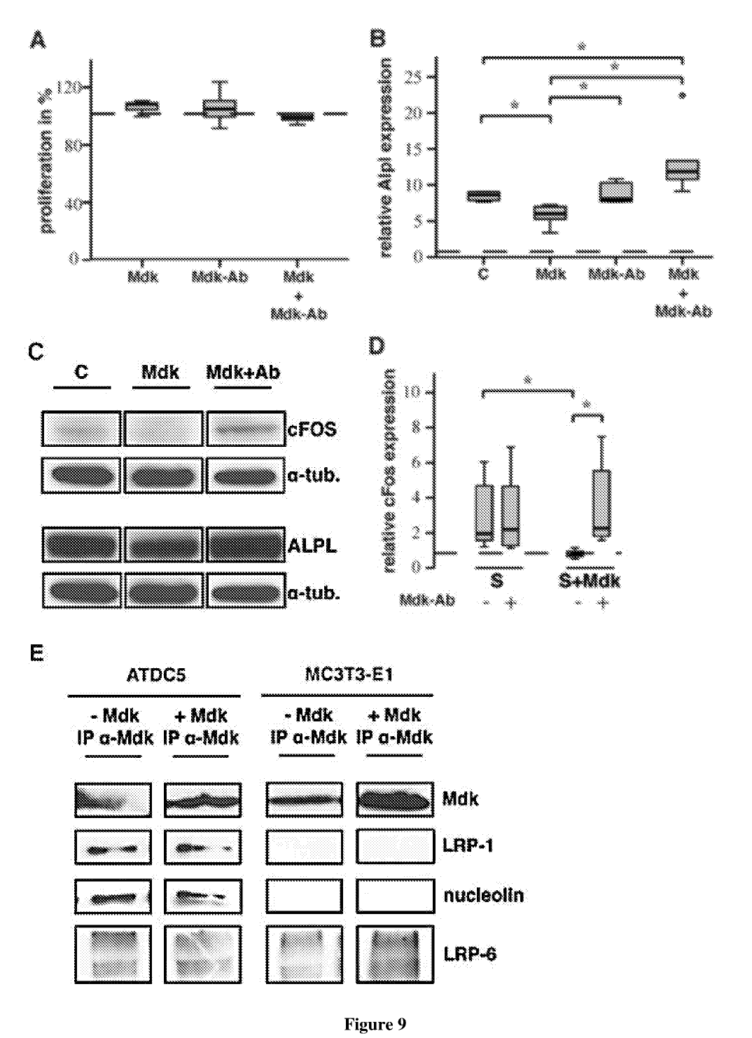

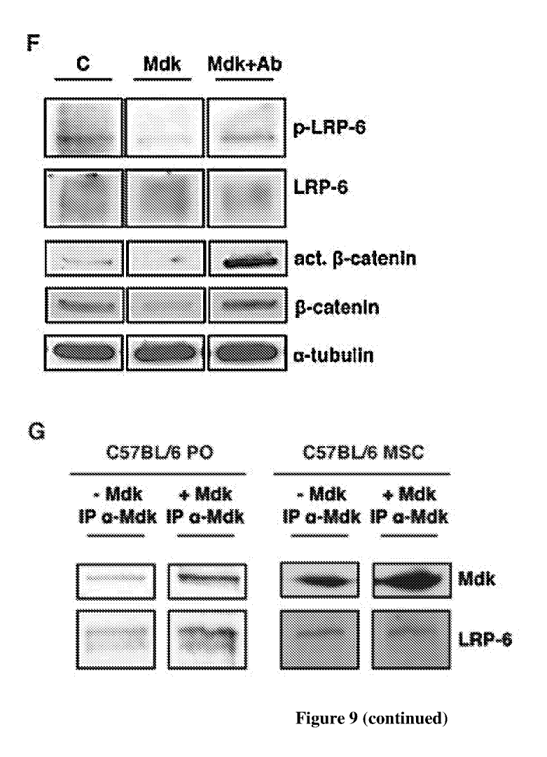

The effect of midkine protein and the anti-midkine antibody on the proliferation of MC3T3-E1 cells was evaluated using MTT assay. No differences were observed between the groups after 6 h of midkine protein or anti-midkine antibody stimulation (FIG. 9A). However, at day 5 of differentiation, stimulation with recombinant midkine protein significantly downregulated alkaline phosphatase (Alpl) and cFOS expression (FIGS. 9, B and C). Additional treatment with the anti-midkine antibody abolished the midkine-induced effects.

To analyze the influence of midkine protein on osteoblastic cell response to mechanical stimulation, MC3T3-E1 cells were exposed to cyclic stretching (FIG. 9 D). Recombinant Mdk downregulated the mechanically induced gene expression of cFos by these cells, while additional incubation with anti-midkine antibody significantly attenuated this effect.

To investigate putative midkine receptors during fracture healing, immunoprecipitation was performed using murine chondroprogenic ATDC5 cells and osteoprogenic MC3T3-E1 cells stimulated with midkine protein. Interestingly, the previously described intracellular midkine-interacting proteins LRP-1 and nucleolin (Sakamoto et al., (2011) J. Biol. Chem., 286:8405-13; Lee et al., (2012) J. Cell Physiol. 227:1731-9; Take et al., (1994) J. Biochem. 16:1063-1068) were immunoprecipitated with midkine protein only in ATDC5 cells, whereas the canonical Wnt-signaling receptor LRP-6 (Li et al., (2002) J. Biol. Chem. 277:5977-81) was immunoprecipitated with midkine protein in both cell types (FIG. 9 E). The amount of precipitated LRP-6 protein was increased by exogenous midkine protein in MC3T3-E1 cells (FIG. 9E). Based on this finding, the role of LRP-6 in midkine-induced effects on these cells was investigated as described above. It was shown that LRP-6 phosphorylation was decreased after 6 h of midkine protein stimulation (FIG. 9F). Additionally, total beta-catenin and active beta-catenin protein expression were decreased following midkine protein stimulation, these effects being attenuated by anti-midkine antibody.

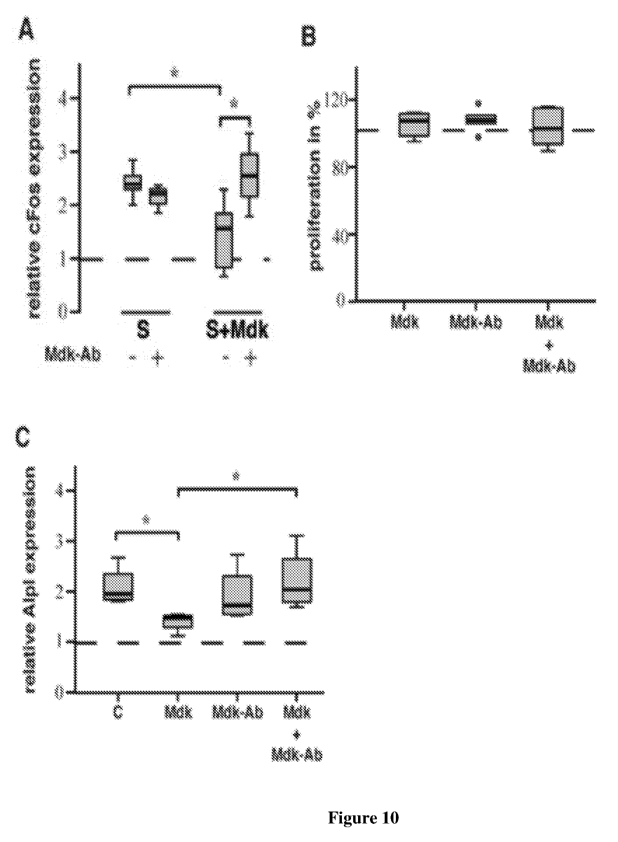

Next, midkine protein immunoprecipitation was performed in calvarian primary osteoblasts and mMSCs isolated from C57BL/6 mice. Again, it was shown that LRP-6 coimmunoprecipitates with midkine protein (FIG. 9G). Based on this finding, the effects of midkine protein and the anti-midkine antibody on primary cells was assessed. It was demonstrated that recombinant midkine protein treatment in primary osteoblasts also downregulated mechanically induced cFos gene expression, while additional incubation with anti-midkine antibody significantly attenuated this effect (FIG. 10A). Incubation of mMSCs with recombinant midkine protein did no alter the proliferation rate (FIG. 10), whereas Alpl expression was decreased (FIG. 10C), which was similarly abolished by treatment with an anti-midkine antibody.

### Example 4—Effect of Treatment with Anti-Midkine Antibody on Early Immune Response and Fracture Healing in Oestrogen-Deficient Mice

Fracture healing is known to be delayed in post-menopausal, osteoporotic females under oestrogen-deficient conditions. Oestrogen-deficiency is also known to affect the immune system and the inflammatory response during wound healing. Since a balanced immune response is required for proper bone healing, the inventors sought to determine the effect of oestrogen depletion on the early immune response after facture in mice. The inventors hypothesised that oestrogen-deficiency increases early immune response after fracture and that inflammatory mediators, such as midkine (which is a pro-inflammatory cytokine and negative regulator of bone remodeling), may be involved in this effect. To test this hypothesis, the inventors analysed the presence of immune cells and inflammatory cytokines in the fracture haematoma of OVX-mice and sham-operated mice. The inventors also determined the effect of treatment with anti-midkine antibody on early immune response and fracture healing in OVX-mice and sham-operated mice.

### Methods

### Animals

All mice used in this experiment were female C57BL/6J mice provided by the University of Ulm. The mice were maintained in groups of two to four animals per cage (370 cm²) on a 14 h light and 10 h dark circadian rhythm with water and phytoestrogen-free diet ad libitum for the entirety of the experiment.

### Treatment

Briefly, three to four month old mice were randomly divided into two groups that underwent a bilateral sham-operation or ovariectomy (OVX), respectively, in accordance with the method described in Example 2. Eight weeks after receiving the sham operation or OVX, animals received a standardized osteotomy at the midshaft of the right femur using a 0.4 mm Gigli saw (RISystem, Davos, Switzerland) stabilized using an external fixator (axial stiffness of 3.0 N/mm, RISystem). Immediately following the osteotomy, treatment was initiated to evaluate the effect of midkine on the fracture callus. During treatment, half the animals in the sham-operated group and half the animals in the OVX group received anti-midkine antibody, IP-10, which was administered subcutaneously at 25 mg/kg twice weekly for 3 weeks. The remaining animals in the sham-operated and OVX groups were treated in parallel using the vehicle phosphate-buffered saline (PBS). Animals from each of the four groups were sacrificed on days 0, 3 or 23 post-osteotomy using carbon dioxide (n=5-6 per group at each time point). The fractured and intact femurs, as well as the uteri, were removed from all mice for further analysis.

### Micro-Computed Tomography (μCT)

Femurs of mice sacrificed on day 23 were analyzed using a μCT scanning device (Skyscan 1172, Kontich, Belgium) operating at a voxel resolution of 8 μm (50 kV, 200 mA). Bone mineral density (BMD) was assessed using two phantoms, each with a defined hydroxyapatite density (250 mg/cm³ and 750 mg/cm³) within each scan. Discrimination between non-mineralised and mineralised tissue was performed using a global threshold of 642 mg hydroxyapatite/cm³ as described in Morgan et al., (2009) Bone, 44(2):225-244 and in accordance with the guidelines set forth by the American Society for Bone and Mineral Research (ASBMR) for μCT analysis as described in Bouxsein et al., (2010) J. Bone Miner. Res., 25(7):1468-1486. Three-dimensional (3D) reconstruction of the fracture callus between the two inner pin holes was performed using CTvol software (Bruker).

### Fluorescent-Activated Cell Sorting (FACS) Analysis

To analyse inflammatory cells in the fracture haematoma, FACS analysis was performed. On day 1 post-osteotomy, the fractured femur and the contralateral bone marrow were harvested. The fracture haematoma was collected using a surgical scissor. The contralateral bone marrow was flushed out using PBS. The fracture haematoma was passed through a 70-μm cell strainer (Corning Inc., Durham, N.C.) to obtain a single-cell suspension and the cells of the fracture haematoma and bone marrow were subjected to erythrolysis. The antibodies listed in Table 3 were used for the identification of macrophages (Ly6G⁻, F4/80⁺, CD11b+), neutrophils (Ly-6G⁺, F4/80⁻, CD11b⁺), inflammatory monocytes (F4/80⁺, Ly-6G⁺, CD11b⁺), B-lymphocytes (CD19⁺), T-lymphocytes (CD3⁺), cytotoxic T-lymphocytes (CD3⁺, CD8⁺), and T-helper-lymphocytes (CD3⁺, CD4⁺). Specific isotype-matched immunoglobulin antibodies (Table 3) were used as negative controls. Cells (fracture haematoma: totality of cells isolated; bone marrow: 1×10⁶ cells) were incubated with the antibodies for 30 minutes on ice. 7-aminoactinomycin (7-AAD, Sigma, Steinheim, Germany) was used for dead-cell discrimination. Cells were analysed using an LSR II flow cytometer (BD Bioscience) and FlowJo software v10 (FlowJo LLC, Ashland, Oreg.).

TABLE 3
Antibodies used for flow cytometry

| Antibody | Label | Product | Company | Dilution |
|---|---|---|---|---|
| Il-6 (rabbit anti-mouse) | — | bs-0782R | Bioss | 1:250 |
| Mdk (goat anti-mouse) | — | sc-1398 | SantaCruz | 1:100 |
| CCL2 (rabbit | — | bs-1955R | Bioss | 1:150 |
| anti-mouse) | | | | |
| CXCL1 (rabbit | — | ab86436 | Abcam | 1:200 |
| anti-mouse) | | | | |
| Ly6G (rat anti-mouse) | — | 127603 | BioLegend | 1:300 |
| CD45/B220 (rat | — | RA3-6B2 | BioLegend | 1:100 |
| anti-mouse) | | | | |
| CD8 (rabbit | — | bs-0648R | Bioss | 1:100 |
| anti-mouse) | | | | |
| F4/80 (rat anti-mouse) | — | ab6640 | AbD Serotec | 1:500 |
| Streptavidin | HRP | ZUC012 | Zytomed Sytems | 1:100 |
| IgG (donkey anti-goat) | biotin | sc-3854 | SantaCruz | 1:100 |
| IgG (goat anti-rabbit) | biotin | B2770 | Life Technologies | 1:100 |
| IgG (goat anti-rat) | biotin | 31830 | Invitrogen | 1:100 |

### Histomorphometry of Decalcified Femora and Immunohistochemistry

Femurs of mice sacrificed 3 or 10 days post-osteotomy were fixed in 4% formalin, decalcified using 20% ethylenediaminetetraacetic acid (pH 7.2-7.4) for 10-12 days and embedded in paraffin after dehydration in ascending ethanol series. Longitudinal cross-sections of 7 μm thickness were prepared. Immunohistochemical and immunofluorescence staining of IL-6, Midkine, CCL2, CXCL1, Ly6G (neutrophils), CD45R (B-lymphocytes), CD8 (cytotoxic T-lymphocytes) and F4/80 (macrophages) were performed using the antibodies specified in Table 4. Species-specific non-targeting immunoglobulins were used as isotype controls. 3-Amino-9-ethylcarbazol (Zytomed Systems) was used as the chromogen and the sections were counterstained using hematoxylin (Waldeck, Münster, Germany). FITC-streptavidin was used for immunofluorescence staining and the sections were counterstained using DAPI. The sections were examined by light or fluorescence microscopy (DMI6000 B, Leica, Heerbrugg, Switzerland). The amount of callus tissue and the number of positively stained cells were determined by image analysis software (Leica MMAF 1.4.0 Imaging System, Leica).

TABLE 4
Antibodies used for immunohistochemistry

| Antibody | Label | Product | Company | Dilution |
|---|---|---|---|---|
| CD3e (Armenian | PE-Cyanine 7 | 25-0031 | Affymetrics eBioscience | 1:100 |
| hamster anti-mouse) | | | | |
| CD4 (rat anti-mouse) | APC-eFlour ® 780 | 47-0041 | Affymetrics eBioscience | 1:200 |
| CD8a (rat anti-mouse) | APC | 17-0081 | Affymetrics eBioscience | 1:800 |
| CD11b (rat anti-mouse) | Alexa Flour ® 700 | 56-0112 | Affymetrics eBioscience | 1:400 |
| CD19b (rat anti-mouse) | PE | 12-0193 | Affymetrics eBioscience | 1:400 |
| F4/80 (rat anti-mouse) | FITC | 11-4701 | Affymetrics eBioscience | 1:50 |
| Ly-6G (rat anti-mouse) | V450 | 560603 | BD Bioscience | 1:400 |
| IgG Isotype (Armenian | PE-Cyanine 7 | 25-4888 | Affymetrics eBioscience | 1:100 |
| hamster) | | | | |
| IgG2b K Isotype (rat) | APC-eFlour ® 780 | 47-4031 | Affymetrics eBioscience | 1:200 |
| IgG2a K Isotype (rat) | APC | 17-4321 | Affymetrics eBioscience | 1:800 |
| IgG2b K Isotype (rat) | Alexa Flour ® 700 | 56-4031 | Affymetrics eBioscience | 1:400 |
| IgG2a K Isotype (rat) | PE | 12-4321 | Affymetrics eBioscience | 1:400 |
| IgG2a K Isotype (rat) | FITC | 11-4321 | Affymetrics eBioscience | 1:50 |
| IgG2a K Isotype (rat) | V450 | 560377 | BD Bioscience | 1:400 |

### Statistical Analysis

Statistical analysis was performed using Shapiro-Wilk test for normal distribution and ANOVA/LSD post hoc test with SPSS software (SPSS Inc., Chicago, Ill.). All results are presented as the mean and standard deviation. Values of p<0.05 were considered as statistically significant. Sample size was n=5-6 per group at each time point.

### Results

### OVX-Induced Osteoporosis and Delayed Fracture Healing

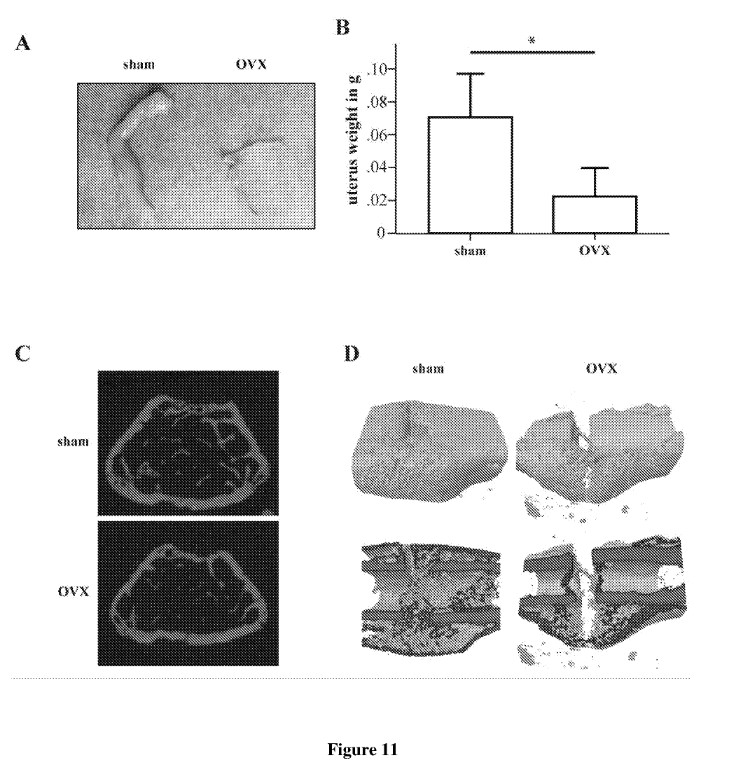

As expected, uteri of mice subjected to ovariectomy displayed severe atrophy (FIG. 11A) and a significantly decreased weight (FIG. 11B). μCT analysis of the intact femurs demonstrated an osteoporotic phenotype in the trabecular compartment (FIG. 11C). Analysis of the fracture calli on day 23 showed considerably disturbed bony callus development and poor cortical bridging due to OVX (FIG. 11D).

### OVX Increased the Number of Neutrophils in the Fracture Haematoma

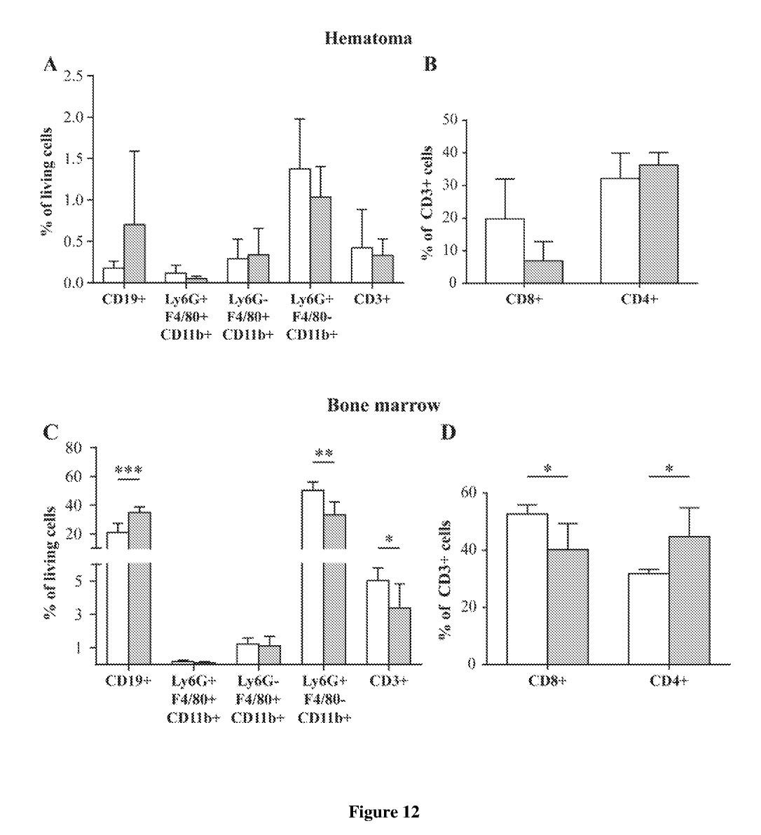

The presence of immune cells in the fracture haematoma on day one after surgery as determined by FACS analysis is shown in FIGS. 12A and B. The cell populations of the innate and adaptive immune systems did not differ significantly between sham-operated and OVX-mice at this time point at the fracture site. However, OVX-mice significantly increased the number of B-lymphocytes and reduced the number of T-lymphocytes in the bone marrow (FIG. 12C). Within the T-lymphocyte population, cytotoxic T-cells were significantly reduced whereas T-helper cells were significantly increased (FIG. 12D). Furthermore, the number of inflammatory monocytes and macrophages were not affected, whereas the number of neutrophils was significantly increased.

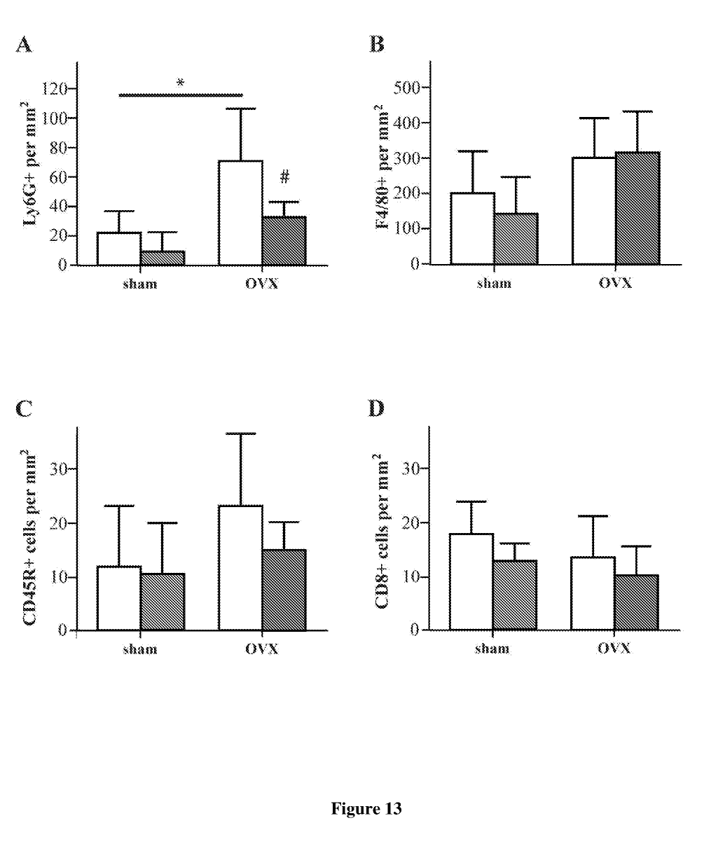

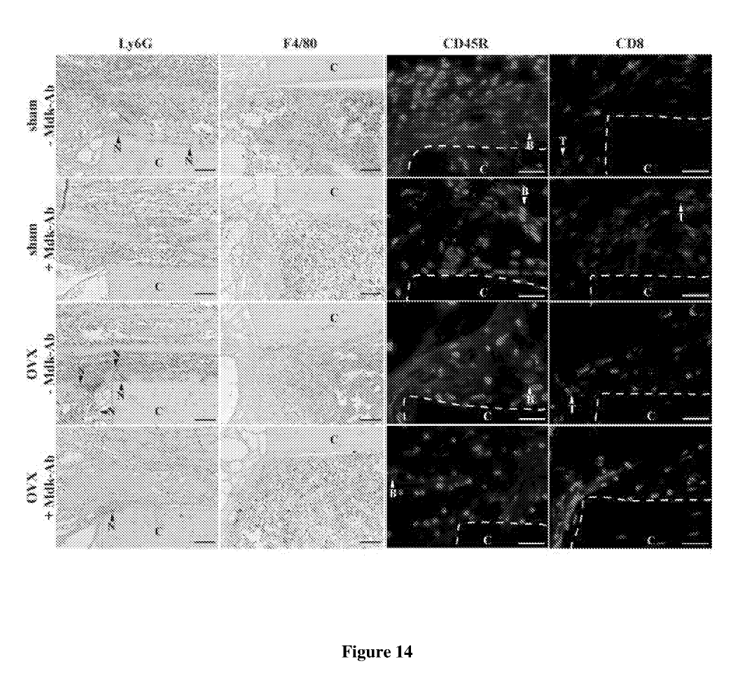

Three days after fracture, immune cells in the fracture callus were evaluated by immunohistochemistry. OVX-mice displayed significantly greater numbers of neutrophils in the periosteal callus (FIG. 13A). Neutrophils were mainly found in the fracture gap surrounding fibrous tissue as well as in the early periosteal fracture callus (FIG. 14). The number of macrophages, which were mainly located in the bone marrow near the osteotomy gap, did not differ significantly between the groups (FIG. 13B). There were no differences in the number of B-lymphocytes or cytotoxic T-lymphocytes, which were located in the periosteal fracture callus (FIGS. 13C and D).

### OVX Increased Midkine and IL-6 Expression in the Fracture Callus

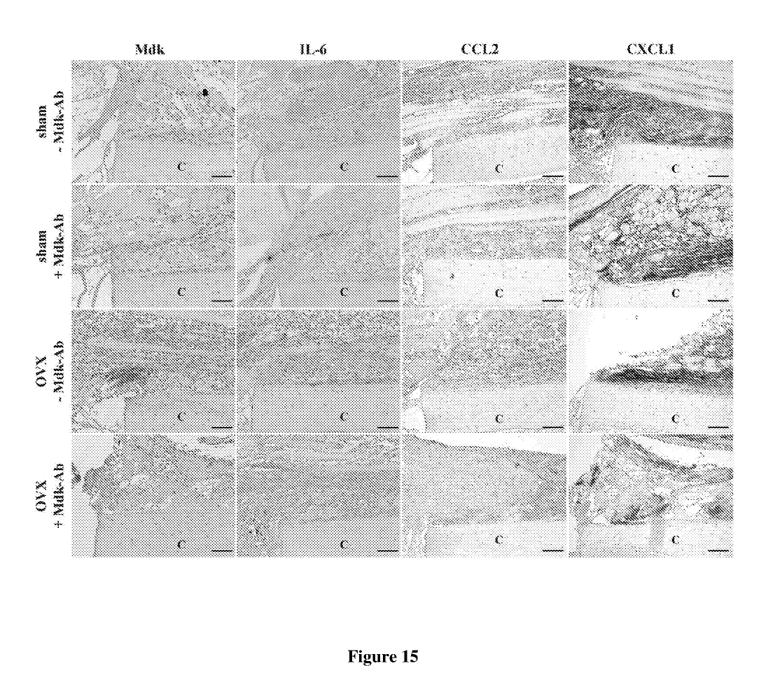

The role of midkine during the inflammatory phase of fracture healing was investigated by analysing the local expression of midkine in the callus by immunostaining. On day 3, midkine was expressed in the fracture callus of OVX-mice (FIG. 15). The protein was located at the periosteal transition zone from haematoma tissue to soft fracture callus proximal to the osteotomy gap. Midkine was expressed less in sham-operated animals.

Since it is known that midkine expression is associated with IL-6 expression and because IL-6 is a potent inductor for neutrophil recruitment, IL-6 expression was also evaluated by immunostaining. In sham-operated mice, IL-6 was expressed in muscle tissue and endosteal cells, but was expressed less in periosteal cells, whereas OVX-mice showed increased expression in periosteal cells adjacent to the fracture gap (FIG. 15).

Since it was hypothesized that the up-regulation of midkine expression after OVX may be involved in increased neutrophil numbers in the periosteal callus, the effect of treatment with an inhibitory midkine antibody was evaluated. Mice treated with an inhibitory midkine antibody directly after surgery displayed significantly lower numbers of neutrophils in the fracture callus compared to non-treated mice (FIG. 13A), whereas the number of macrophages, B-lymphocytes and cytotoxic T-lymphocytes was not significantly altered (FIG. 13B-D). Furthermore, OVX-mice treated with an inhibitory midkine antibody exhibited lower IL-6 expression in the periosteal cells next to the fracture gap (FIG. 15). Protein expression of the proinflammatory cytokines CCL2 and CXCL1 were similar between the groups (FIG. 15).

### CONCLUSIONS

The results of this study demonstrated that numbers of B-lymphocytes in the bone marrow were elevated in OVX-mice 1 day after fracture, whereas the number of bone marrow neutrophils and total T-lymphocytes were reduced but with an increased ratio of CD4⁺/CD8⁺ cells. Since B-lymphocytes are known to synthesize several inflammatory cytokines, as well as recent finding suggesting that they are active regulators of the RANK/RANKL/OPG system, this data supports the conclusion that there is a strong association between increased numbers of B-lymphocytes and bone loss during menopause.

Since B- and T-lymphocytes, as well as neutrophils, affect fracture healing outcome, the number of these cells were also evaluated in the early fracture haematoma. No differences were observed between OVX- and sham-mice on day 1 after trauma, although the cell populations were different in the bone marrow. This finding indicates that the initial recruitment of inflammatory cells to the fracture callus was unaffected by oestrogen-deficiency. However, on day 3 after fracture, significantly more neutrophils were present in the periosteal callus of oestrogen-deficient mice, indicating a prolonged recruitment and/or an increased survival of neutrophils at the fracture site in the absence of oestrogen. These data suggest that the pro-inflammatory cytokine midkine may be involved in the effects of oestrogen-deficiency on the inflammatory phase of fracture healing.

In study described in Example 2, it was shown that midkine serum levels were increased in oestrogen-deficient mice from day 3 to day 23 after fracture. In this study, it was shown that there was increased local expression of midkine in the fracture callus of OVX-mice on day 3 after fracture. Since midkine is known to chemoattract both neutrophils and macrophages, it was suggested that increased midkine expression may be involved in the prolonged presence of neutrophils at the fracture callus in OVX-mice. In support of this conclusion, significantly decreased numbers of neutrophils were observed after treatment with the anti-midkine antibody. However, no significant changes in CXCL1 expression were observed, one of the most important proteins for neutrophil recruitment.

Previous studies have shown that midkine-deficient mice displayed lower numbers of neutrophils and macrophages in the tubulointerstitium after ischaemic renal injury (Sato et al., (2001) J. Immunol., 167:3463-3469) and that midkine-deficiency delayed the recruitment of macrophages to the fracture site during the regenerative phase of healing. However, in the present study, no significant changes in the number of macrophages or the expression of monocyte chemoattractant protein 1 (CCL2) were detected. Additionally, no changes in the number of B- or T-lymphocytes in the fracture callus were detected, although it was demonstrated that midkine regulated B-cell survival in vitro. On this basis, the increased midkine expression in the early fracture callus of oestrogen-deficient mice appears to predominantly affect the recruitment and survival of neutrophils.

In the literature, several pro-inflammatory cytokines are described to be involved in the increased severity of inflammatory disorders in oestrogen-deficient subjects. One of these cytokines is IL-6, which is known as a crucial factor for the recruitment of inflammatory cells (Jones et al., (2006) J. Infect. Dis., 193:360-369; Rose-John S: (2015) Best Pract. Res. Clin. Endocrinol. Metab. 29:787-797). Additionally, several studies have demonstrated increased IL-6 expression after tissue injury in oestrogen-deficient mice (Aydin et al., (2015) Basic Clin. Pharmacol. Toxicol., 117:173-179; Shivers et al., (2015) Cytokine, 72:121-129). In the present study, higher IL-6 expression was observed in the periosteal cells of OVX-mice relative to sham-mice, but was diminished in both sham and OVX mice treated with anti-midkine antibody. On this basis, it was concluded that increased IL-6 expression due to the lack of oestrogen may contribute to the increased number of neutrophils in the fracture callus of OVX-mice and that blocking midkine leads to reduced IL-6 and reversed the recruitment of neutrophils to the fracture site.

In conclusion, this study demonstrates that oestrogen-deficiency significantly influenced the early inflammatory phase after fracture. Higher midkine and IL-6 expression at the fracture site were associated with increased numbers of neutrophils in the callus. Conversely, neutralising midkine with the antibody reduced neutrophils and abundance of IL-6 in parallel with accelerated fracture healing in osteoporotic mice as observed in Example 2.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Kadomatsu. et al. (1988) Biochem. Biophys. Res. Commun., 1511312-1318
Tomokura et al. (1999) J. Biol. Chem, 265:10765-10770
Muramatsu (2002) J. Biochem. 132:359-371
WO2000/10608
WO2004/078210
Iwasaki et al. (1997) EMBO J. 16:6936-6946
WO2008/059616
WO2012/122590
Cadet et al., (2013) J. Am. Acad. Orthop. Surg. 21:538-547
King et al., (2007) Humeral nonunion. Hand Clin. 23:449-456
Einhorn et al., (2015) Nature Reviews Rheumatology, 11:45-54
Hayda et al., (1998) Clin. Orthop. Relat. Res., 355(suppl):31-40
Einhorn T A (2003) J. Bone Joint Surg. Am. 85-A(Suppl 3):82-88
Giannoudis and Dinopoulos (2010) J. Orthop. Trauma, 24(Suppl 1):S9-16
Busse et al., (2002) CMAJ, 166:437-441
Poynton et al., (2002) J. Orthop. Trauma, 24:522-525
J. Perbal, A Practical Guide to Molecular Cloning, John Wiley and Sons (1984)
J. Sambrook et al. Molecular Cloning: A Laboratory Manual, Cold Spring Harbour Laboratory Press (1989)
T. A. Brown (editor), Essential Molecular Biology: A Practical Approach, Volumes 1 and 2, IRL Press (1991)
D. M. Glover and B. D. Hames (editors), DNA Cloning: A Practical Approach, Volumes 1-4, IRL Press (1995 and 1996)
F. M. Ausubel et al. (editors), Current Protocols in Molecular Biology, Greene Pub. Associates and Wiley-Interscience (1988, including all updates until present)
Ed Harlow and David Lane (editors) Antibodies: A Laboratory Manual, Cold Spring Harbour Laboratory, (1988)
J. E. Coligan et al. (editors) Current Protocols in Immunology, John Wiley & Sons (including all updates until present)
Kabat Sequences of Proteins of Immunological Interest, National Institutes of Health, Bethesda, Md., 1987 and 1991
Bork et al., (1994) J. Mol. Biol. 242:309-320
Chothia and Lesk (1987) J. Mol. Biol. 196:901-917
Chothia et al. (1989) Nature 342:877-883
Al-Lazikani et al., (1997) J. Mol. Biol. 273:927-948.
WO1994/007921
WO1998/044001
Lefranc et al., (2003) Dev. Comp. Immunol., 27(1)55-77
WO2008/059616
WO2012/122590
WO2014/070642
Sun X. Z, et al., (1997) J. Neuropathol. Exp. Neurol. 56(12):1339-48
Muramatsu H., et al., (2004) J. Biochem., 119:1171-77
Needleman and Wunsch, (1970) J. Mol. Biol., 48(3):443-453
Rich and Myszka, (2000) Curr. Opin. Biotechnol. 11:54;
Englebienne P, (1998) Analyst. 123(7):1599-1603
Malmqvist M (1999), Biochem. Soc. Trans. 27:335-340
U.S. Pat. No. 4,676,980
WO1991/000360
WO1992/200373
EP 586505
Caron et al., (1992) J. Exp. Med., 176(4):1191-1195
Shopes B. (1992) J. Immunol., 148(9):2918-2922
Stevenson et al., (1989) JAMA, 261:884-888
U.S. Pat. No. 4,196,265
WO2002/066630
Largaespada et al, (1996) J. Immunol. Methods. 197:85-95
U.S. Pat. No. 6,300,064
U.S. Pat. No. 5,885,793
U.S. Pat. No. 4,816,567
U.S. Pat. No. 5,807,715
U.S. Pat. No. 5,225,539
U.S. Pat. No. 5,585,089
U.S. Pat. No. 5,565,332
WO2000/034317
WO2004/108158
WO2004/064724
WO1994/004678
WO1997/049805
WO2005/118629
WO1998/044001
WO1994/007921
US20060263367
U.S. Pat. No. 7,235,641
U.S. Pat. No. 4,816,567
U.S. Pat. No. 5,530,101
WO1999/057134
US20060228364
US20080260757
U.S. Pat. No. 6,306,610
Remington's Pharmaceutical Sciences (Mack Publishing Co. N.J. USA, 1991
Röntgen et al., (2010) J. Orthop. Res., 28:1456-1462
Morgan et al., (2009) Bone, 44(2):225-244
Wehrle et al., (2014) J. Orthop. Res., 32(8):1006-1013
O'Neill et al, (2012) Bone, 50:1357-67
Bouxsein et al., (2010) J. Bone Miner. Res., 25(7):1468-1486
Heilmann et al., (2013) PLoS One, 8(12):e84232
Liedert et al., (2011) Bone, 48:945-951
Haffner-Luntzer et al., (2014) PLoS One, 9(12):e116282
Schmidt et al., (2005) J. Cell Biol., 168:899-910
Huebner et al., (2006) J. Bone Miner. Res. 21:1924-1934
Kaspar et al., (2000) J. Biomech. 33:45-51
Ramakers et al., (2003) Neuroscience Letters, 339:62-66
Sakamoto et al., (2011) J. Biol. Chem., 286:8405-13
Lee et al., (2012) J. Cell Physiol. 227:1731-9
Take et al., (1994) J. Biochem. 16:1063-1068
Sato et al., (2001) J. Immunol., 167:3463-3469
Jones et al., (2006) J. Infect. Dis., 193:360-369
Rose-John S: (2015) Best Pract. Res. Clin. Endocrinol. Metab. 29:787-797
Aydin et al., (2015) Basic Clin. Pharmacol. Toxicol., 117:173-179
Shivers et al., (2015) Cytokine, 72:121-129

1. A method of promoting bone formation and/or promoting bone healing and/or increasing bone mineral density (BMD) in a subject, said method comprising administering to the subject an isolated or recombinant protein comprising an antigen binding domain of an antibody which binds specifically to midkine (MK) protein.

2. The method according to claim 1, wherein the subject is selected from the groups consisting of:
(i) a subject with a bone fracture;
(ii) a subject who has undergone, or will undergo, a surgical procedure to create bone;
(iii) a subject who has undergone, or will undergo, a surgical procedure to promote integration of an orthopedic implant or hardware with adjacent bone; and
(iv) a subject suffering from or predisposed to osteoporosis.

3. The method according to claim 1 or claim 2, wherein administration of the isolated or recombinant protein accelerates bone healing and/or enhances osteoblast activity in the subject.

4. The method according to any one of claims 1 to 3, wherein the isolated or recombinant protein binds specifically to an epitope located within the N-domain of MK and inhibits or reduces a function of MK.

5. The method according to claim 4, wherein the epitope to which the isolated or recombinant protein binds is located within the N-domain of MK as defined by amino acid residues 1-61 of the sequence set forth in SEQ ID NO: 1.

6. The method according to any one of claims 1 to 5 wherein the isolated or recombinant protein recognizes at least a portion of a high electrostatic potential cluster located at amino acid residues 1-61 of the sequence set forth in SEQ ID NO: 1.

7. The method according to any one of claims 1 to 6, wherein the isolated or recombinant protein binds specifically to a conformational epitope formed by the amino acid sequence set forth in SEQ ID NO:1, wherein the epitope includes at least two residues selected from the group consisting of 18W, 20W, 34F, 35R, 36E, 38T, 43T, 45R, 47R and 49R.

8. The method according to claim 7, wherein the epitope is defined by the following residues:
(i) 18W, 20W, 35R and 49R;
(ii) 18W, 20W, 36E, 38T, 43T and 45R; or
(iii) 18W, 20W, 34F, 36E, 45R and 47R.

9. The method according to any one of claims 1 to 8, wherein the isolated or recombinant protein comprises:
(i) a heavy chain variable domain (V_{H}) comprising the sequence set forth in SEQ ID NO: 2 or a sequence exhibiting 95% or greater identity thereto;
(ii) a light chain variable domain (V_{L}) comprising the sequence set forth in SEQ ID NO: 3 or a sequence exhibiting 95% or greater identity thereto;
(iii) a V_{H} comprising the sequence set forth in SEQ ID NO: 2 or a sequence exhibiting 95% or greater identity thereto and a V_{L} comprising the sequence set forth in SEQ ID NO: 3 or a sequence exhibiting 95% or greater identity thereto;
(iv) a V_{H} comprising CDR1, CDR2 and CDR3 comprising the sequences set forth in SEQ ID NO: 4, 5 and 6 respectively, and a V_{L} comprising CDR1, CDR2 and CDR3 comprising the sequences set forth in SEQ ID NO: 7, 8 and 9 respectively; or
(v) three CDRs comprised within the sequence set forth in SEQ ID NO: 2 and three CDRs comprised within the sequence set forth in SEQ ID NO: 3.

10. The method according to any one of claims 1 to 8, wherein the isolated or recombinant protein comprises:
(i) a heavy chain variable domain (V_{H}) comprising the sequence set forth in SEQ ID NO: 10 or a sequence exhibiting 95% or greater identity thereto;
(ii) a light chain variable domain (V_{L}) comprising the sequence set forth in SEQ ID NO: 11 or a sequence exhibiting 95% or greater identity thereto;
(iii) a V_{H} comprising the sequence set forth in SEQ ID NO: 10 or a sequence exhibiting 95% or greater identity thereto and a V_{L} comprising the sequence set forth in SEQ ID NO: 11 or a sequence exhibiting 95% or greater identity thereto;
(iv) a V_{H} comprising CDR1, CDR2 and CDR3 comprising the sequences set forth in SEQ ID NO: 12, 13 and 14 respectively, and a V_{L} comprising CDR1, CDR2 and CDR3 comprising the sequences set forth in SEQ ID NO: 15, 16 and 17 respectively; or
(v) three CDRs comprised within the sequence set forth in SEQ ID NO: 10 and three CDRs comprised within the sequence set forth in SEQ ID NO: 11.

11. The method according to any one of claims 1 to 8, wherein the isolated or recombinant protein comprises:
(i) a heavy chain variable domain (V_{H}) comprising the sequence set forth in SEQ ID NO: 18 or a sequence exhibiting 95% or greater identity thereto;
(ii) a light chain variable domain (V_{L}) comprising the sequence set forth in SEQ ID NO: 19 or 20 or a sequence exhibiting 95% or greater identity thereto;
(iii) a V_{H} comprising the sequence set forth in SEQ ID NO: 18 or a sequence exhibiting 95% or greater identity thereto and a V_{L} comprising the sequence set forth in SEQ ID NO: 19 or 20 or a sequence exhibiting 95% or greater identity thereto;
(iv) a V_{H} comprising CDR1, CDR2 and CDR3 comprising the sequences set forth in SEQ ID NO: 21, 22 and 23 respectively, and a V_{L} comprising CDR1, CDR2 and CDR3 comprising the sequences set forth in SEQ ID NO: 24, 25 and 26 respectively; or
(v) three CDRs comprised within the sequence set forth in SEQ ID NO: 18 and three CDRs comprised within the sequence set forth in SEQ ID NO: 19 or 20.

12. The method according to any one of claims 1 to 3, wherein the isolated or recombinant protein binds specifically to an epitope located within the C-domain of MK and inhibits or reduces a function of MK.

13. The method according to claim 12, wherein the epitope to which the isolated or recombinant protein binds is located within the C-domain of MK as defined by amino acid residues 62-104 of the sequence set forth in SEQ ID NO:1.

14. The method according to any one of claims 1 to 3 or 12 or 13, wherein the epitope to which the isolated or recombinant protein binds is located within amino acid residues 64-73 and amino acid residues 78-101 of the sequence set forth in SEQ ID NO:1.

15. The method according to any one of claims 1 to 3 or 12 to 14, wherein the isolated or recombinant protein recognizes at least a portion of an epitope located at amino acid residues 64 to 66, amino acid residues 64 to 67, amino acid residues 64 to 69, amino acid residues 64 to 73, amino acid residues 84 to 96, or amino acid residues 87 to 96 of the sequence set forth in SEQ ID NO: 1.

16. The method according to any one of claims 1 to 3 or 12 to 15, wherein the isolated or recombinant protein binds specifically to an epitope formed by the amino acid sequence set forth in SEQ ID NO:1, wherein the epitope includes at least one residues selected from the group consisting of 64Y 65K, 66F, 67E, 69W, 73D, 84T, 86K, 87K 90Y and 96E.

17. The method according to any one of claims 12 to 16, wherein the epitope is conformation epitope.

18. The method according to claim 17, wherein the conformational epitope is an antiparallel β-sheet epitope.

19. The method according to any one of claims 1 to 3 or 12 or claim 13, wherein the isolated or recombinant protein recognizes at least a portion of a high electrostatic potential cluster located at amino acid residues 62-104 of the sequence set forth in SEQ ID NO: 1.

20. The method according to claim 19, wherein the isolated or recombinant protein recognizes at least one amino acid selected from the group consisting of amino acid residues 62-64, 66, 68-70, 72, 79, 81, 85-89, 102 and 103 of the sequence set forth in SEQ ID NO:1.

21. The method according to claim 19 or claim 20, wherein the isolated or recombinant protein binds specifically to an epitope formed by the amino acid sequence set forth in SEQ ID NO: 1, wherein the epitope includes at least one residues selected from the group consisting of 63K, 79K, 81R 86K, 87K, 89R and 102K.

22. The method according to any one of claims 1 to 3 or 12 to 21, wherein the isolated or recombinant protein comprises:
(i) a heavy chain variable domain (V_{H}) comprising CDR1, CDR2 and CDR3 comprising the sequences set forth in SEQ ID NO: 27, 28 and 29 respectively; and
(ii) a light chain variable domain (V_{L}) comprising CDR1, CDR2 and CDR3 comprising the sequences set forth in SEQ ID NO: 30, 31 and 32 respectively.

23. The method according to any one of claims 1 to 3 or 12 to 21, wherein the isolated or recombinant protein comprises:
(i) a heavy chain variable domain (V_{H}) comprising CDR1, CDR2 and CDR3 comprising the sequences set forth in SEQ ID NO: 33, 34 and 35 respectively; and
(ii) a light chain variable domain (V_{L}) comprising CDR1, CDR2 and CDR3 comprising the sequences set forth in SEQ ID NO: 36, 37 and 38 respectively.

24. The method according to any one of claims 1 to 23 wherein the isolated or recombinant protein comprises a heavy chain variable domain (VH) and a light chain variable domain (VL).

25. The method according to claim 22 or claim 23, wherein the VH and the VL are in a single polypeptide chain.

26. The method according to claim 25, wherein the isolated or recombinant protein is:
(i) a single chain Fv fragment (scFv);
(ii) a dimeric scFv (di-scFv); or
(iii) at least one of (i) and/or (ii) linked to a Fc or a heavy chain constant domain (CH) 2 and/or CH3.

27. The method according to claim 22 or claim 23, wherein the VL and VH are in separate polypeptide chains.

28. The method according to claim 27, wherein the isolated or recombinant protein is:
(i) a diabody;
(ii) a triabody;
(iii) a tetrabody;
(iv) a Fab;
(v) a F(ab′)2;
(vi) a Fv; or
(iv) one of (i) to (iii) linked to a Fc or a heavy chain constant domain (CH) 2 and/or CH3.

29. The method according to any one of claims 1 to 28 wherein the isolated or recombinant protein is a chimeric, de-immunized, humanized or human antibody.

30. The method according to any one of claims 1 to 29 wherein the isolated or recombinant protein comprises a human or non-human primate heavy chain immunoglobulin constant region selected from a group consisting of IgG, IgG2, IgG3, IgG4, IgM, IgE and IgA.

31. The method according to any one of claims 1 to 30 wherein the isolated or recombinant protein is conjugated to a compound.

32. The method according to claim 31, wherein the compound is selected from the group consisting of a radioisotope, a detectable label, a therapeutic compound, a colloid, a toxin, a nucleic acid, a peptide, a protein, a compound that increases the half-life of the protein in a subject and mixtures thereof.


SEQUENCE LISTING (38 sequences)

SEQ ID NO: 1 - protein, 121 aa, Homo sapiens
KKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 2 - protein, 120 aa, Artificial sequence
  IP9 Heavy chain variable region
QVQLQQPGTELVKPGASVKLSCKASGYTFTSYWMHWVKQRPGHGLDWIGNINPHNGGTNYNEKFNNRATLTVDKSSTTAYMQLSSLTSEDSADYYCVRGGHYDEGYFEYWGQGTTLTVSS

SEQ ID NO: 3 - protein, 107 aa, Artificial sequence
  IP9 Light chain variable region
DIQMTQSPASLSASVGETVTITCRASGNIHNYLAWYQQKQGKSPQFLVYNAKTLADGVPSRFSGSGSGTQFSLKINSLQPEDFGNYYCQHFWSTPFTFGSGTKLEIK

SEQ ID NO: 4 - protein, 8 aa, Artificial sequence
  IP9 VH CDR1
GYTFTSYW

SEQ ID NO: 5 - protein, 8 aa, Artificial sequence
  IP9 VH CDR2
INPHNGGT

SEQ ID NO: 6 - protein, 13 aa, Artificial sequence
  IP9 VH CDR3
VRGGHYDEGYFEY

SEQ ID NO: 7 - protein, 9 aa, Artificial sequence
  IP9 VL CDR1
DIQMTQSPA

SEQ ID NO: 8 - protein, 6 aa, Artificial sequence
  IP9 VL CDR2
GNIHNY

SEQ ID NO: 9 - protein, 3 aa, Artificial sequence
  IP9 VL CDR3
NAK

SEQ ID NO: 10 - protein, 121 aa, Artificial sequence
  IP10 Heavy chain variable region
EVQLQQSGPELVKPGDSVKISCKASGYSFTGYFMNWVMQSHGKSLEWIGRINPYNGDTFYNQQFKGKATLTVDKSSSTAHMELRSLTSEDSAVFYCAREAGGSSFDWLAYWGRGTLVTVSA

SEQ ID NO: 11 - protein, 107 aa, Artificial sequence
  IP10 Light chain variable region
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 12 - protein, 8 aa, Artificial sequence
  IP10 VH CDR1
GYSFTGYF

SEQ ID NO: 13 - protein, 8 aa, Artificial sequence
  IP10 VH CDR2
INPYNGDT

SEQ ID NO: 14 - protein, 14 aa, Artificial sequence
  IP10 VH CDR3
AREAGGSSFDWLAY

SEQ ID NO: 15 - protein, 6 aa, Artificial sequence
  IP10 VL CDR1
ENVVTY

SEQ ID NO: 16 - protein, 3 aa, Artificial sequence
  IP10 VL CDR2
GAS

SEQ ID NO: 17 - protein, 9 aa, Artificial sequence
  IP10 VL CDR3
GQGYSYPYT

SEQ ID NO: 18 - protein, 119 aa, Artificial sequence
  IP13 Heavy chain variable region
EVQLQQSGPELVKPGASVKIPCKASGYTFTDYNMGWVKQSHGKSLEWIGDINPNNGGTIYNQKFKGKATLTVDKSSSTAYMELRSLTSEDTAVYYCARWSYSNPYFDYWGQGTTLTVSS

SEQ ID NO: 19 - protein, 107 aa, Artificial sequence; identical to SEQ ID NO: 11
  IP13 Light chain variable region v1

SEQ ID NO: 20 - protein, 107 aa, Artificial sequence
  IP13 Light chain variable region v2
SIVMTQTPNFLLVSAGDRVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 21 - protein, 8 aa, Artificial sequence
  IP13 VH CDR1
GYTFTDYN

SEQ ID NO: 22 - protein, 8 aa, Artificial sequence
  IP13 VH CDR2
INPNNGGT

SEQ ID NO: 23 - protein, 12 aa, Artificial sequence
  IP13 VH CDR3
ARWSYSNPYFDY

SEQ ID NO: 24 - protein, 6 aa, Artificial sequence
  IP13 VL CDR1
ENVVTY

SEQ ID NO: 25 - protein, 3 aa, Artificial sequence
  IP13 VL CDR2
GAS

SEQ ID NO: 26 - protein, 9 aa, Artificial sequence
  IP13 VL CDR3
GQGYSYPYT

SEQ ID NO: 27 - protein, 5 aa, Artificial sequence
  CSM-1 VH CDR1
DYFLH

SEQ ID NO: 28 - protein, 17 aa, Artificial sequence
  CSM-1 VH CDR2
RIDPEDSETKYAPKFQG

SEQ ID NO: 29 - protein, 9 aa, Artificial sequence
  CSM-1 VH CDR3
NYGGGGFAY

SEQ ID NO: 30 - protein, 11 aa, Artificial sequence
  CSM-1 VL CDR1
KASQDVSTAVA

SEQ ID NO: 31 - protein, 7 aa, Artificial sequence
  CSM-1 VL CDR2
SASYRYT

SEQ ID NO: 32 - protein, 9 aa, Artificial sequence
  CSM-1 VL CDR3
QQHYSSPFT

SEQ ID NO: 33 - protein, 5 aa, Artificial sequence
  CSM-4 VH CDR1
SYWMN

SEQ ID NO: 34 - protein, 17 aa, Artificial sequence
  CSM-4 VH CDR2
MIHPSDSETILNQKFKD

SEQ ID NO: 35 - protein, 8 aa, Artificial sequence
  CSM-4 VH CDR3
WSAKRGDF

SEQ ID NO: 36 - protein, 5 aa, Artificial sequence
  CSM-4 VL CDR1
SYWMN

SEQ ID NO: 37 - protein, 7 aa, Artificial sequence
  CSM-4 VL CDR2
YASQSIS

SEQ ID NO: 38 - protein, 9 aa, Artificial sequence
  CSM-4 VL CDR3
QQSNSWPLT